(19) United States
(12) Patent Application Publication
(10) Pub. No.: US 2014/0065241 A1
(43) Pub. Date: Mar. 6, 2014

(54) USES OF (-)-PERHEXILINE

(71) Applicants: Adelaide Research & Innovation Pty Ltd, Adelaide (AU); ITEK Ventures Pty Ltd, Salisbury South (AU); Central Adelaide Local Health Network Inc., Adelaide (AU)

(72) Inventors: Benedetta SALLUSTIO, Manningham, South Australia (AU); Robert MILNE, Maylands, South Australia (AU); John LICARI, West Lakes, South Australia (AU); Andrew Alexander SOMOGYI, Athelstone, South Australia (AU)

(73) Assignees: Adelaide Research & Innovation Pty Ltd, Adelaide (AU); ITEK Ventures Pty Ltd, Salisbury South (AU); Central Adelaide Local Health Network Inc., Adelaide (AU)

(21) Appl. No.: 14/019,207

(22) Filed: Sep. 5, 2013

Related U.S. Application Data
(60) Provisional application No. 61/697,214, filed on Sep. 5, 2012.

(30) Foreign Application Priority Data
Sep. 5, 2012 (AU) 2012903850

(51) Int. Cl. A61K 31/4458; A61K 31/7004; G01N 33/50; A61K 38/26; A61K 45/06; A61K 38/28; A61K 33/00

(52) U.S. Cl. CPC A61K 31/4458; A61K 38/28; A61K 31/7004; A61K 33/00; A61K 38/26; A61K 45/06; G01N 33/5008
USPC 424/600; 514/317; 514/6.5; 514/23; 514/7.2; 435/29

(57) ABSTRACT

A method for treating or preventing a disease, condition or state in a subject in need thereof by administering to the subject an effective amount of the (−)-enantiomer of perhexyline or a pharmaceutically acceptable salt, prodrug or derivative thereof, substantially free of the (+)-enantiomer, or pharmaceutical composition containing same. The disease, condition or state may be associated with altered tissue energetics, impaired tissue energetics, altered cardiac tissue energetics, impaired cardiac tissue energetics, altered hepatic tissue energetics, impaired hepatic tissue energetics, and diabetes.

### CROSS-REFERENCE TO RELATED APPLICATIONS

This application is related to, and claims the benefit of, (1) U.S. Provisional Patent Application No. 61/697,214, filed on Sep. 5, 2012; (2) Australian Provisional Patent Application No. 2012903850 filed on Sep. 5, 2012; and (3) PCT/International Patent Application No. [not yet assigned] (bearing Docket No. P1130901), which was electronically filed concurrently herewith in the Australian Patent Office (as the Receiving Office) on Sep. 5, 2013, and assigned Electronic Submission No. SPEC-19069461 (Applicants hereby reserve the right to insert the ultimately assigned International Patent Application No. upon receipt of same from the Receiving Office). The entire contents of each of the above-identified patent applications are incorporated herein by reference.

### BACKGROUND

The exemplary embodiments disclosed herein generally relate to the use of the (−)-enantiomer of perhexyline (also known as 2-(2,2-dicyclohexylethyl)piperidine) for preventing and/or treating a disease, condition or state associated with altered or impaired tissue energetics, including, but not limited to, altered myocardial metabolism associated with cardiovascular related disease states.

It has become increasingly apparent that many diseases, conditions and states are the direct or indirect result of perturbed tissue energy metabolism. For example, altered myocardial metabolism is increasingly recognised as an underlying defect in many forms of cardiovascular disease. Altered energy metabolism is also associated with diseases such as diabetes and metabolic syndrome.

Cardiovascular diseases, such as ischaemic heart disease and heart failure, impose a significant cost burden on the health systems in most developed countries. Ischaemic heart disease manifests clinically as angina or myocardial infarction, and is a leading cause of heart failure. Ischaemic heart disease is the leading cause of mortality and morbidity in most developed countries, and a large proportion of patients are disabled by ischaemic heart disease, requiring assistance with daily living. Furthermore, the ageing of populations, improved survival rates of myocardial infarction and increasing rates of diabetes and obesity are also leading to an increasing prevalence of heart failure.

Current therapies for ischaemic heart disease generally involve haemodynamic changes to improve the imbalance between myocardial oxygen supply and demand. However, many patients continue to experience symptoms despite maximal conventional medication. Therapies for heart failure, such as angiotensin-converting enzyme (ACE) inhibitors, beta-blockers, aldosterone antagonists and diuretics are beneficial for systolic heart failure, but have a variety of deficiencies and have also not made a significant impact in the treatment of diastolic heart failure.

Because altered myocardial metabolism has become increasingly recognised as an underlying defect in many forms of cardiovascular disease, including ischaemic heart disease and heart failure, metabolic agents that improve myocardial energetics are emerging as an important new therapeutic class for cardiovascular diseases.

Perhexyline is a metabolic agent that has been used as an anti-anginal agent. Perhexyline is a chiral compound and is used clinically as a racemic (50:50) mixture of (+)- and (−)-enantiomers. It is believed that perhexyline reduces fatty acid metabolism through the inhibition of carnitine palmitoyltransferase 1, the enzyme responsible for mitochondrial uptake of long-chain fatty acids. The corresponding shift to greater carbohydrate utilization increases myocardial efficiency (work done per unit oxygen consumption) and this oxygen-sparing effect explains its antianginal efficacy.

However, the clinical use of perhexyline has been limited, in large part due to its severe adverse effects, the need to maintain a systemic/plasma concentration within a narrow range and its complex metabolism which manifests in high inter- and intra-individual pharmacokinetic variability. The adverse effects of perhexyline are typically seen in more than 60% of recipients. The most commonly reported minor adverse effects include headache, dizziness, nausea and vomiting. The severe adverse effects include hepatotoxicity and peripheral neuropathy, typically seen at plasma concentrations greater than 0.6 mg/L. Perhexyline's side effects are particularly apparent in patients with impaired metabolism due to CYP2D6 mutation. However, dose modification in these poorly metabolizing patients identified through therapeutic plasma monitoring can be used to minimise the side effects.

One way to characterize a chemical composition containing a compound having at least one chiral center is by the effect of the composition on a beam of polarized light. When a beam of plane polarized light is passed through a solution of a chiral compound, the plane of polarization of the light that emerges is rotated relative to the original plane. This phenomenon is known as optical activity, and compounds that rotate the plane of polarized light are said to be optically active. One enantiomer of a compound will rotate the beam of polarized light in one direction, and the other enantiomer will rotate the beam of light in the opposite direction. The enantiomer that rotates the polarized light in the clockwise direction is the (+)-enantiomer and the enantiomer that rotates the polarized light in the counterclockwise direction is the (−)-enantiomer.

Stereochemical purity is sometimes important for biologically active substances that are used in pharmaceutical compositions for human application since the respective enantiomers may have a different potency or may have a different activity. Often, one of the enantiomers presents the desired optimum biological activity. Additionally, the presence of the other enantiomer in a composition or agent may cause or exacerbate certain side effects. It is sometimes desirable to administer the biologically active substance in the form of a substantially pure enantiomer, which specifically exhibits a desired biological activity. Therefore, the resolution of a racemate into its enantiomers is often an important step in the preparation process of pharmacologically active substances. In some cases, enantiomers of a compound have genuinely different effects. In other cases, there may be no pharmaceutical or clinical benefit. And it is possible that both enantiomers are active or only one of them is active. These factors cannot be determined or predicted ahead of time without the rigors of experimental study.

With respect to perhexyline, for example, previous studies suggest that the (+)-enantiomer of perhexyline is the main contributor to the clinical efficacy of racemic perhexyline because it has been observed to be present in higher plasma concentrations compared to the (−)-enantiomer of perhexyline. In addition, the (+)-enantiomer of perhexyline is not as effectively metabolized as the (−)-enantiomer. Previous studies have also suggested that the plasma concentration of (+)-perhexyline is much less dependent on or affected by genetic variability as compared to the (−)-enantiomer of perhexyline. Still, it is believed that the pharmacokinetics profile of resolved perhexyline enantiomers is not a reliable predictor of better clinical efficacy or even less severe adverse side effects of (−)-perhexyline over (+)-perhexyline, and vice versa.

Despite the severe adverse effects of perhexyline, the racemic drug is still believed to hold a critically important place in Australia and New Zealand for the treatment of patients with refractory angina or those who have contraindications to other standard anti-anginal therapies. Perhexyline is also increasingly being used for the treatment of acute coronary syndromes. However, this requires close monitoring of plasma concentrations to maintain the systemic/plasma concentration within a strict or narrow range to manage the clinical toxicity of the drug.

It would be advantageous to use perhexyline, or derivatives of perhexyline, but with reduced severe adverse effects and/or without a need to maintain strict monitoring and control over systemic/plasma concentrations. In some circumstances, it would be advantageous to use lower doses of perhexyline or derivatives thereof. Accordingly, there is a need to address the aforementioned and other problems relating to the use of perhexyline.

### SUMMARY OF EXAMPLE EMBODIMENTS

Certain exemplary embodiments disclosed herein are based, in part, on an unpredicted/unexpected recognition that hepatotoxicity and/or neuropathy observed in connection with the administration of racemic perhexyline are associated with/caused by the (+)-enantiomer and not the (−)-enantiomer. Further, it has been unexpectedly discovered that administration of the (+)-enantiomer is associated with increased hepatic lipid content and decreased hepatic glycogen, and that administration of the (−)-enantiomer conversely is associated with increased hepatic glycogen but no apparent effect on hepatic lipid content. As a result, it is now believed that (+)-perhexyline causes hepatic steatosis at plasma concentrations similar to those causing hepatotoxicity in humans. It is further believed that (+)-perhexyline causes a significant decline in neural sensory function, whereas (−)-perhexyline does not. With respect to heart failure, it has been discovered that the (−)-enantiomer is actually more effective in preventing cardiac damage than the (+)-enantiomer, while both enantiomers are believed to retain similar anti-inflammatory potential and ability to reduce oxidative stress. The (−)-enantiomer is also believed to be a much more potent inhibitor of pyruvate dehydrogenase phosphorylation (i.e., deactivation) compared to the (+)-enantiomer, indicating that the (−)-enantiomer is associated with more efficient carbohydrate utilization, and therefore clinically useful as a metabolic agent.

Certain example embodiments of the present disclosure provide a method for preventing and/or treating a disease, condition or state associated with altered or impaired tissue energetics in a subject in need thereof, the method comprising administering to the subject an effective amount of the (−)-enantiomer of perhexyline and/or a pharmaceutically acceptable salt, prodrug or derivative thereof, substantially free of the other enantiomer.

Other exemplary embodiments of the present disclosure provide use of substantially enantiopure (−)-perhexyline and/or a pharmaceutically acceptable salt, prodrug or derivative thereof in the manufacture of a medicament for preventing and/or treating a disease, condition or state associated with altered tissue energetics.

Certain further embodiments of the present disclosure provide a method for preventing and/or treating a disease, condition or state associated with impaired cardiac tissue energetics in a subject in need thereof, the method comprising administering to the subject an effective amount of substantially enantiopure (−)-perhexyline and/or a pharmaceutically acceptable salt, prodrug or derivative thereof.

Certain embodiments of the present disclosure provide use of substantially enantiopure (−)-perhexyline, and/or a pharmaceutically acceptable salt, prodrug or derivative thereof in the manufacture of a medicament for preventing and/or treating a disease, condition or state associated with impaired cardiac tissue energetics in a subject.

Certain embodiments of the present disclosure provide a method for preventing and/or treating a subject in need thereof of ischaemic heart disease, heart failure including systolic and diastolic heart failure, angina, refractory angina, ventricular hypertrophy, cardiomyopathy including congestive cardiomyopathy and hypertrophic cardiomyopathy, the method administering to the subject an effective amount of substantially enantiopure (−)-perhexyline and/or a pharmaceutically acceptable salt, prodrug or derivative thereof.

Certain embodiments of the present disclosure provide use of substantially enantiopure (−)-perhexyline, and/or a pharmaceutically acceptable salt, prodrug or derivative thereof in the manufacture of a medicament for preventing and/or treating in a subject one of more of ischaemic heart disease, heart failure including systolic and diastolic heart failure, angina, refractory angina, ventricular hypertrophy, cardiomyopathy including congestive cardiomyopathy and hypertrophic cardiomyopathy.

Certain embodiments of the present disclosure provide use of substantially enantiopure (−)-perhexyline, and/or a pharmaceutically acceptable salt, prodrug or derivative thereof in the manufacture of a medicament for preventing and/or treating type I or type II diabetes in a subject in need thereof.

Certain embodiments of the present disclosure provide a method for preventing and/or treating type I or type II diabetes in a subject, the method administering to the subject an effective amount of substantially enantiopure (−)-perhexyline and/or a pharmaceutically acceptable salt, prodrug or derivative thereof.

Certain embodiments of the present disclosure provide a method for administration of perhexyline with reduced adverse effects, the method comprising administering to a subject in need thereof an effective amount of substantially enantiopure (−)-perhexyline and/or a pharmaceutically acceptable salt, prodrug or derivative thereof.

Certain embodiments of the present disclosure provide a method for reducing one or more adverse effects in a subject associated with administration of perhexyline, the method comprising administering to the subject an effective amount of substantially enantiopure (−)-perhexyline and/or a pharmaceutically acceptable salt, prodrug or derivative thereof.

Certain embodiments of the present disclosure provide use of substantially enantiopure (−)-perhexyline and/or a pharmaceutically acceptable salt, prodrug or derivative thereof in the manufacture of a medicament for reducing one or more adverse effects in a subject in need thereof associated with perhexyline administration.

Certain embodiments of the present disclosure provide use of substantially enantiopure (−)-perhexyline and/or a pharmaceutically acceptable salt, prodrug or derivative thereof in the manufacture of a medicament with reduced adverse effects.

Certain embodiments of the present disclosure provide a pharmaceutical composition comprising substantially enantiopure (−)-perhexyline and/or a pharmaceutically acceptable salt, prodrug or derivative thereof.

Certain embodiments of the present disclose provide a method of reducing cardiac damage in a subject susceptible to, or suffering from, one or more of ischaemic heart disease, heart failure including systolic and diastolic heart failure, angina, refractory angina, ventricular hypertrophy, cardiomyopathy including congestive cardiomyopathy and hypertrophic cardiomyopathy, the method comprising administering to the subject an effective amount of substantially enantiopure (−)-perhexyline and/or a pharmaceutically acceptable salt, prodrug or derivative thereof.

Certain embodiments of the present disclosure provide a method for screening for an agent for preventing and/or treating a disease, condition or state associated with altered tissue energetics, the method comprising:
selecting a modified form of (−)-perhexyline; and
identifying the modified form of (−)-perhexyline as an agent for preventing and/or treating a disease, condition or state associated with altered tissue energetics.

Certain embodiments of the present disclosure provide a method for screening for a cardiac metabolic agent with reduced hepatotoxicity and/or reduced neuropathy, the method comprising:
selecting a derivative of (−)-perhexyline,
identifying the derivative of (−)-perhexyline as an agent that increases hepatic carbohydrate metabolism without substantially increasing hepatic fatty acid metabolism, and
identifying the derivative of (−)-perhexyline as a cardiac metabolic agent with reduced hepatotoxicity and/or reduced neuropathy.

Certain embodiments of the present disclosure provide a treatment regime with reduced adverse effects for treating a disease, condition or state associated with altered tissue energetics, the treatment regime comprising:
administering to a subject in need thereof an effective amount of (−)-enantiomer of perhexyline and/or a pharmaceutically acceptable salt, prodrug or derivative thereof, substantially free of the other enantiomer; and
optionally administering one or more other compounds for treating the disease, condition or state.

Certain embodiments of the present disclosure provide a method for identifying a subject suitable for treatment with (−)-perhexyline, the method comprising identifying a subject with one or more of the following characteristics: ischaemic heart disease, heart failure including systolic and diastolic heart failure, angina, refractory angina, ventricular hypertrophy, cardiomyopathy including congestive cardiomyopathy and hypertrophic cardiomyopathy, type I or type II diabetes, increased tissue NADH/NAD+, reduced tissue pyruvate dehydrogenase activity, increased anaerobic glycolysis, increased fatty acid β-oxidation, reduced phosphocreatine concentration, reduced oxidative phosphorylation, increased insulin resistance, and a reduced ratio of phosphocreatine to ATP.

Certain embodiments of the present disclosure provide a method for optimizing therapeutic efficacy of (−)-perhexyline, the method comprising:
administering (−)-perhexyline to a subject in need thereof;
determining a level of the (−)-perhexyline in the subject that is less than a first predetermined level corresponding to a second predetermined amount; and
increasing the amount of (−)-perhexyline subsequently administered to the subject.

Certain embodiments of the present disclosure provide a method for optimizing therapeutic efficacy of (−)-perhexyline, the method comprising:
administering (−)-perhexyline to a subject in need thereof;
determining a level of the (−)-perhexyline in the subject that is greater than a first
predetermined level corresponding to a second predetermined amount; and
decreasing the amount of (−)-perhexyline subsequently administered to the subject.

The exemplary embodiments will be better understood and appreciated in conjunction with the following detailed description taken together with the accompanying figures.

### BRIEF DESCRIPTION OF THE FIGURES

It is to be understood that the following description of the figures is for the purpose of describing example embodiments only and is not intended to be limiting with respect to this disclosure.

FIG. 1 shows plasma troponin T concentrations in control (CONT) Dark Agouti rats and those injected with isoprenaline (50 mg/kg) alone (ISO) or following 2 weeks pre-treatment with 200 mg/kg/day of (+)-, (−)- or racemic-perhexyline. *=P<0.05, **=P<0.01, ***=P<0.001 c.f. CONT, +++=P<0.001 c.f. ISO

FIG. 2 shows histological assessment of mean (sd) hepatic lipid and glycogen content (% field area) in Dark Agouti rats (n=4) treated with vehicle (Cont), racemic (Rac), (+)- or (−)-perhexyline for 8 weeks (*p<0.05 vs Cont).

FIG. 3 shows linear regression analyses of hepatic (+)-perhexyline concentration versus A) lipid or B) glycogen contents, and C) hepatic (−)-perhexyline concentration versus glycogen content. Data are pooled from control animals and those treated with pure enantiomer or racemate.

FIG. 4 shows the mean (sd) paw withdrawal thresholds (g) in Dark Agouti rats (n=4) treated for 8 weeks with vehicle (Cont), racemic (Rac), (+)- or (−)-perhexyline (*p<0.05 vs Cont).

FIG. 5 shows the concentration-dependent inhibition of human neutrophil NOX2 activity by (+)- and (−)-perhexyline, as determined by measurement of percentage inhibition of superoxide formation.

FIG. 6 shows A: liver (open bars), heart (hatched bars) and plasma (closed bars) concentrations of (+)- and (−)-perhexyline following 8 weeks administration of 200 mg/kg/day of the pure enantiomers or the racemate (concentrations are shown adjusted for an equivalent enantiomeric dose of 100 mg/kg); and B: the corresponding tissue to plasma concentration ratios for liver (open bars) and heart (hatched bars). ^{#}P<0.05 c.f. (+)-PX in liver; *p<0.05 c.f. (+)-PX in heart; ^{%}p<0.05 c.f. R-(+)-PX in liver; ^{$}p<0.05 c.f. R-(+)-PX in plasma; ^{@}p<0.05 c.f. R (+)-Px in heart.

FIG. 7 shows semi-quantitative histological scoring of myocardial damage in control (CONT) Dark Agouti rats and those injected with isoprenaline (50 mg/kg) alone (ISO) or following 2 weeks pre-treatment with 200 mg/kg/day of (+)-, (−)- or racemic-perhexyline. Data are divided into animals scoring 0-1 (no or mild) or >1 (moderate to severe) as described in the text. *=P<0.05 Chi-squared c.f. ISO; **=P<0.05 c.f. CONT.

FIG. 8 shows the extent of TXNIP staining (as a % of the field of view) following immunohistochemistry of heart tissues from control (CONT) Dark Agouti rats and those injected with isoprenaline (50 mg/kg) alone (ISO) or following 2 weeks pre-treatment with 200 mg/kg/day of (+)-, (−)- or racemic-perhexyline. ⁺=P<0.05, ⁺⁺=P<0.01 c.f. CONT; *=P<0.05 c.f. ISO; ̂=P=0.05 c.f. ISO.

FIG. 9 shows in panel A total pyruvate dehydrogenase proteins content and in panel B phosphorylated pyruvate dehydrogenase content in control (CONT) Dark Agouti rats and those injected with isoprenaline (50 mg/kg) alone (ISO) or following 2 weeks pre-treatment with 200 mg/kg/day of (+)-, (−)- or racemic-perhexyline. *p<0.05, **p<0.01, ***p<0.001 c.f. ISO

### DETAILED DESCRIPTION OF EXEMPLARY EMBODIMENTS

To the extent necessary to provide descriptive support, the subject matter and/or text of the appended claims is incorporated herein by reference in their entirety. It will be understood by all readers of this written description that the exemplary embodiments described and claimed herein may be suitably practiced in the absence of any recited feature, element or step that is, or is not, specifically disclosed herein.

All publications and references cited herein, including those in the background section, are expressly incorporated herein by reference in their entirety. However, with respect to any similar or identical terms found in both the incorporated publications or references and those explicitly put forth or defined in this document, then those terms definitions or meanings explicitly put forth in this document shall control in all respects. Further, any reference to prior art in this specification is not, and should not be taken as, an acknowledgment or any form of suggestion that this prior art forms part of the common general knowledge in any country.

The example embodiments disclosed herein relate, in part, to the use of substantially pure (−)-enantiomer of perhexyline for preventing and/or treating a disease condition or state associated with altered tissue energetics and to the identification of agents for preventing and/or treating a disease condition or state associated with altered tissue energetics.

To facilitate understanding of this disclosure set forth herein, a number of terms are defined below. Generally, the nomenclature used herein and the laboratory procedures in organic chemistry, medicinal chemistry, and pharmacology described herein are generally well known and commonly employed in the art. Unless defined otherwise, all technical and scientific terms used herein generally have the same meaning as commonly understood in the art to which this disclosure belongs. In the event that there is a plurality of definitions for a term used herein, those in this section prevail unless stated otherwise.

As used herein, the singular forms “a,” “an,” and “the” may refer to plural articles unless specifically stated otherwise.

Recitation of ranges of values herein are merely intended to serve as a shorthand method of referring individually to each separate value falling within the range, unless otherwise indicated herein, and each separate value is incorporated into the specification as if it were individually recited herein. Where a specific range of values is provided, it is understood that each intervening value, to the tenth of the unit of the lower limit unless the context clearly dictates otherwise, between the upper and lower limit of that range and any other stated or intervening value in that stated range, is included therein. All smaller subranges are also included. The upper and lower limits of these smaller ranges are also included therein, subject to any specifically excluded limit in the stated range.

The term “about” or “approximately” means an acceptable error for a particular value, which depends in part on how the value is measured or determined. In certain embodiments, “about” can mean 1 or more standard deviations. When the antecedent term “about” is applied to a recited range or value it denotes an approximation within the deviation in the range or value known or expected in the art from the measurements method. For removal of doubt, it shall be understood that any range stated herein that does not specifically recite the term “about” before the range or before any value within the stated range inherently includes such term to encompass the approximation within the deviation noted above.

The term “preventing”, and related terms such as “prevention” and “prevent”, refer to obtaining a desired pharmacologic and/or physiologic effect in terms of delaying, precluding, arresting or suppressing the appearance of one or more symptoms in a subject and/or reducing the risk of the subject from acquiring a disorder.

The term “treatment”, and related terms such as “treating” and “treat”, refer to obtaining a pharmacologic and/or physiologic effect in terms of improving the condition of a subject, abrogating, alleviating, ameliorating, arresting, suppressing, relieving, preventing and/or slowing the progression or cause of one or more symptoms in the subject, a partial or complete stabilization of the subject, a regression of the one or more symptoms, or a cure of a disease, condition or state in the subject.

The term “subject” refers to an animal, including, but not limited to, a primate (e.g., human, monkey, chimpanzee, gorilla, and the like), rodents (e.g., rats, mice, gerbils, hamsters, ferrets, and the like), lagomorphs, swine (e.g., pig, miniature pig), equine, canine, feline, and the like. The terms “subject” and “patient” are used interchangeably herein in reference, for example, to a mammalian subject, such as a human patient.

The term “modified form of perhexyline” as used in reference to screening methods refers to a chemical, structural, isomeric or stereoisomeric derivative of perhexyline.

Certain embodiments disclosed herein may have one or more combinations of advantages. For example, some of the advantages of the embodiments disclosed herein may include one or more of the following: improved methods for preventing and/or treating a disease, condition or state associated with altered tissue energetics; methods for preventing and/or treating diseases, conditions or states with reduced adverse effects and/or toxicity associated with racemic perhexyline administration; the ability to use lower doses of perhexyline to prevent and/or treat a disease, condition or state associated with altered tissue energetics; improved pharmaceutical formulations of perhexyline; the ability to administer perhexyline with reduced reliance on monitoring and/or controlling concentrations of the drug; improved reliability of prevention and/or treatment using the drug; improved tolerance of the drug in different types of responders; an improved safety profile in subjects with reduced CYP2D6 activity and in subjects with proficient CYP2D6 activity; improved ability to identify subjects suitable for administration of perhexyline; new methods for identifying metabolic agents for use in the prevention and/or treatment of disease, condition or state associated with altered tissue energetics; the ability to control adherence to medication management or predictive analytics by enhancing, for example, patient specific or stratified data sets used to predict patience adherence to realize greater return on investment; to provide one or more advantages in the art; or to provide a useful commercial choice. Other advantages of certain embodiments are disclosed herein or may be appreciated in practicing one or more embodiments.

Included within the scope of the example embodiments described herein are therapeutic or pharmaceutical compositions, and uses of such compositions, containing between about 0% and 100% of the (−)-enantiomer of perhexyline and all subranges therebetween. In one exemplary embodiment, the present disclosure relates to the use of substantially enantiomerically pure (“enantiopure”) negative isomer of perhexyline, and in particular, to the use of substantially enantiopure (−)-perhexyline for preventing and/or treating a disease, condition or state associated with altered tissue energetics. The present disclosure also relates to pharmaceutical compositions comprising substantially enantiopure (−)-perhexyline and to the identification of agents for preventing and/or treating a disease, condition or state associated with altered tissue energetics.

Any techniques for the preparation/isolation of individual perhexyline compound enantiomers may be used including chiral synthesis from a suitable optically pure precursor or resolution of the racemate using, for example, chiral chromatography, recrystallization, resolution, diastereomeric salt formation, or derivatization into diastereomeric adducts followed by separation. Other methods may also be employed to separate enantiomers such as the classic technique of chiral acid precipitation, which is described in applications EP 828,702 and WO 00/32554 and U.S. Pat. No. 4,571,424, which are hereby incorporated by reference in their entirety. Still other methods known to one of skill in the art and routine modifications thereof, and/or following procedures similar to those described herein and routine modifications thereof, and/or procedures found in, for example, Davies et al., J Chrom B, 832 (2006) 114-120; Jaques et al., Tetrahedron Letters, 48 (1971) 4617-4620; PolInitz et al., Journal of Agricultural and Food Chemistry 2004, 52, 3244-3252, Gayen, Internet Electronic Journal of Molecular Design 2005, 4, 556-578, Shirley et al., J. Amer. Chem. Soc. 1957, 3481-3485, Morimoto J. Med. Chem. 2001, 44, 3355-3368, Esaki Tetrahedron 2006, 62, 10954-10961, Kogon, Organic Syntheses 1963, Collective Volume 4, 182, U.S. Pat. No. 5,883,254, Caton, J. Chem. Soc. C 1967, 13, 1204, U.S. Pat. No. 5,025,031, Harada, Bioorganic & Medicinal Chemistry 2001, 9, 2955-2968, U.S. Pat. No. 5,292,740, and Hopfgartner et al, J. Mass. Spectrom. 1996, 37, 69-76, and references cited therein and routine modifications thereof.

One of skill in the art will appreciate that although the present disclosure exemplifies embodiments of compositions, and use of such compositions, containing a substantially enantiomerically pure isomer of the perhexyline compound, the disclosure contemplates other example embodiments comprising the use of stereoisomeric mixtures of perhexyline but that still achieve advantageous therapeutic effects compared to racemic (50:50) mixtures of perhexyline, depending on one or more of the various factors described herein. In one aspect, for example, exemplary embodiments may include compositions that contain perhexyline compound comprising about 60% or more by weight of the (−)-enantiomer and about 40% or less by weight of (+)-enantiomer; about 70% or more by weight of the (−)-enantiomer and about 30% or less by weight of the (+)-enantiomer; about 80% or more by weight of the (−)-enantiomer and about 20% or less by weight of the (+)-enantiomer; about 90% or more by weight of the (−)-enantiomer and about 10% or less by weight of the (+)-enantiomer; about 95% or more by weight of the (−)-enantiomer and about 5% or less by weight of the (+)-enantiomer; about 99% or more by weight of the (−)-enantiomer and about 1% or less by weight of the (+)-enantiomer; and all subranges therebetween.

Certain embodiments of the present disclosure provide a method for preventing and/or treating a disease, condition or state associated with altered tissue energetics in a subject in need thereof.

Certain embodiments of the present disclosure provide a method for preventing and/or treating a disease, condition or state associated with altered tissue energetics in a subject in need thereof, the method comprising administering to the subject an effective amount of the (−)-enantiomer of perhexyline and/or a pharmaceutically acceptable salt, prodrug or derivative thereof, substantially free of the other enantiomer.

In certain embodiments, the disease, condition or state associated with altered tissue energetics comprises a disease conditions or state associated with impaired tissue energetics, reduced tissue energetics, dysfunctional tissue energetics, altered substrate uptake, storage and/or utilization, and/or altered ATP, phosphocreatine synthesis, storage and/or utilization.

In certain embodiments, the disease, condition or state associated with altered tissue energetic comprises a disease, condition or state associated with altered or impaired cardiac tissue energetics.

In certain embodiments, the disease, condition or state comprises one or more of ischaemic heart disease, heart failure including systolic and diastolic heart failure, angina, refractory angina, ventricular hypertrophy, cardiomyopathy including congestive cardiomyopathy and hypertrophic cardiomyopathy.

In certain embodiments, the disease, condition or state is associated with altered hepatic tissue energetics.

In certain embodiments, the disease, condition or state associated with altered tissue energetics is type I or type II diabetes.

In certain embodiments, the subject is a human subject. In certain embodiments, the subject is a mammalian subject, a livestock animal (such as a horse, a cow, a sheep, a goat, a pig), a domestic animal (such as a dog or a cat) and other types of animals, including laboratory animals such as monkeys, rabbits, mice, guinea pigs and gerbils. Veterinary applications of the present disclosure are contemplated.

In certain embodiments, the subject is suffering from a disease, condition or state associated with altered tissue energetics. In certain embodiments, the subject is suffering from a disease, condition or state associated with impaired cardiac tissue energetics.

In certain embodiments, the subject is suffering from a disease, condition or state associated with altered hepatic tissue energetics.

In certain embodiments, the subject is suffering from fatty liver disease and/or steatohepatitis. In this respect, it is envisaged that (−)-perhexyline will be safer to use, or better tolerated, in subjects suffering from fatty liver disease or steatohepatitis (e.g., NASH).

In certain embodiments, the subject is suffering from peripheral neuropathy. In this respect, it is envisaged that (−)-perhexyline will be safer to use, or better tolerated, in subjects suffering from peripheral neuropathy.

In certain embodiments, the subject is suffering from one or more of ischaemic heart disease, heart failure including systolic and diastolic heart failure, angina, refractory angina, ventricular hypertrophy, cardiomyopathy including congestive cardiomyopathy and hypertrophic cardiomyopathy.

In certain embodiments, the subject is suffering from type I or type II diabetes.

In certain embodiments, the subject is susceptible to a disease, condition or state associated with altered tissue energetics. In certain embodiments, the subject is susceptible to a disease, condition or state associated with impaired cardiac tissue energetics.

In certain embodiments, the subject is susceptible to one or more of ischaemic heart disease, heart failure including systolic and diastolic heart failure, angina, refractory angina, ventricular hypertrophy, cardiomyopathy including congestive cardiomyopathy and hypertrophic cardiomyopathy.

In certain embodiments, the subject is susceptible to disease, condition or state associated with altered hepatic tissue energetics.

In certain embodiments, the subject is susceptible to type I or type II diabetes.

In certain embodiments, the subject has an increased risk or likelihood of suffering from a disease, condition or state associated with altered tissue energetics. In certain embodiments, the subject has an increased risk or likelihood of suffering from a disease, condition or state associated with impaired cardiac tissue energetics.

In certain embodiments, the subject has an increased risk or likelihood of suffering from one or more of ischaemic heart disease, heart failure including systolic and diastolic heart failure, angina, refractory angina, ventricular hypertrophy, cardiomyopathy including congestive cardiomyopathy and hypertrophic cardiomyopathy.

In certain embodiments, the subject has an increased risk or likelihood of suffering from a disease, condition or state associated with altered hepatic tissue energetics.

In certain embodiments, the subject has an increased risk or likelihood of suffering from type I or type II diabetes.

In certain embodiments, the subject has one or more of the following characteristics: ischaemia, increased tissue NADH/NAD+, reduced tissue pyruvate dehydrogenase activity, increased anaerobic glycolysis, increased fatty acid β-oxidation, reduced phosphocreatine concentration, reduced oxidative phosphorylation, increased insulin resistance, and a reduced ratio of phosphocreatine to ATP. Methods for testing and/or identifying the aforementioned characteristics are known in the art.

In certain embodiments, the (−)-enantiomer provides an improved safety profile in all subjects, such as for example in subjects with reduced CYP2D6 activity and/or with proficient CYP2D6 activity. In certain embodiments, the (−)-enantiomer provides an improved safety profile in subjects with reduced CYP2D6 activity and/or in subjects with proficient CYP2D6 activity. The accession number for human CYP2D6 Protein is Genbank CAG30316.

In certain embodiments, the subject “in need thereof” has a reduced ability to metabolise perhexyline. In certain embodiments, the subject has reduced CYP2D6 activity. In certain embodiments, the use of (−)-perhexyline provides an improved safety profile in subjects with reduced CYP2D6 activity.

In certain embodiments, the method comprises administering to the subject an effective amount of the (−)-enantiomer of perhexyline and/or a pharmaceutically acceptable salt, prodrug or derivative thereof, substantially free of the other enantiomer.

The term “perhexyline” refers to the chemical compound 2-(2,2-dicyclohexylethyl) piperidine and which has the following chemical structure:

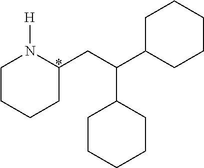 [SMILES: C1CCC(C(CC2CCCCN2)C2CCCCC2)CC1]

Perhexyline exists in two enantiomeric forms about the chiral carbon atom (*), the (+)-enantiomer and the (−)-enantiomer. The enantiomer that rotates the polarized light in the clockwise direction is the (+)-enantiomer and the enantiomer that rotates the polarized light in the counterclockwise direction is the (−)-enantiomer.

The term “perhexyline” is also intended to refer to (in addition to the parent compound) a pharmaceutically acceptable salt of the parent compound (for example the maleate salt, the hydrochloride salt or the lactate salt), a prodrug of the parent compound, a chemical derivative, and/or a stereoisomeric derivative of perhexyline, the aforementioned having substantially the same properties of the parent compound.

In certain embodiments, the perhexyline is substantially enantiopure, being in a form that comprises a single enantiomer substantially free of the other enantiomer.

In certain embodiments, the perhexyline comprises a single enantiomer that comprises greater than 95% of the enantiomer. In certain embodiments, the perhexyline comprises a single enantiomer that comprises greater than about 96%, 97%, 98% or 99% of the enantiomer.

In certain embodiments, the perhexyline comprises a single enantiomer that comprises less than 5% of the other enantiomer. In certain embodiments, the perhexyline comprises a single enantiomer that comprises less than about 4%, 3%, 2% or 1% of the other enantiomer.

Further example embodiments include methods for optimizing the therapeutic efficacy (e.g., by increasing therapeutic effect or reducing adverse side effects or toxicity) of the (−)-enantiomer of perhexyline administered to a subject in need thereof for the treatment of a disorder.

In one example embodiment, the method comprises administering the substantially pure enantiomer of perhexyline to a subject having a disorder treatable by perhexyline (e.g., a disorder indicating a state of altered tissue energetics in the subject); determining or measuring a level of the perhexyline enantiomer in the subject that is less than a predetermined level corresponding to a predetermined amount and increasing the amount of perhexyline enantiomer subsequently administered to the subject.

In another example embodiment, the method comprises administering the substantially pure enantiomer of perhexyline to a subject having a disorder treatable by perhexyline (e.g., a disorder indicating a state of altered tissue energetics in the subject); determining or measuring a level of the perhexyline enantiomer in the subject that is greater than a predetermined level corresponding to a predetermined amount and decreasing the amount of perhexyline enantiomer subsequently administered to the subject.

The concentration level of enantiomer of perhexyline in a treated subject can be determined using any suitable method, for example, plasma or red blood cells using high pressure liquid chromatography or other measuring means. The predetermined amount can be any amount determined by one of skill in the art for treating the disorder as further described herein or known in the art.

In certain embodiments, the administration of the (−)-enantiomer of perhexyline reduces one or more adverse effects as compared to administration of racemic perhexyline.

In certain embodiment, the method for preventing and/or treating a disease, condition or state associated with altered tissue energetics comprises reducing one or more adverse effects in the subject as compared to administration of racemic perhexyline.

In certain embodiments, the one or more adverse effects comprise one or more of hepatotoxicity, neuropathy, and hypoglycaemia. In certain embodiments, the one or more adverse effects comprise hepatotoxicity and/or neuropathy.

In certain embodiments, the administration of the (−)-enantiomer of perhexyline increases hepatic glycogen content without substantially increasing hepatic lipid content and/or non-hepatic tissue lipid content in the subject.

In certain embodiments, the administration of the (−)-perhexyline produces one or more of the following in the subject: increased glucose utilization, increased myocardial lactate utilization, reduced myocardial lactate accumulation, reduced long chain fatty acid utilization, and increased cardiac efficiency.

The term “effective amount” as used herein refers to that amount of an agent (e.g., a perhexyline enantiomer) that is sufficient to effect prevention and/or treatment, when administered to a subject in need thereof and as such is a “therapeutically effective amount.” The effective amount will vary depending upon a number of factors, including for example the specific activity of the agent being used, the severity of the disease, condition or state in the subject, the age, physical condition, existence of other disease states, and nutritional status of the subject. The term “therapeutically effective amount” also refers to the amount of a compound that is sufficient to elicit the biological or medical response of a cell, tissue, system, animal, or human that is being sought by a researcher, veterinarian, medical doctor, or clinician.

The terms “active ingredient,” “active agent” and “active substance” refer to a compound, which is administered, alone or in combination with one or more pharmaceutically acceptable excipients and/or carriers, to a subject for treating, preventing, or ameliorating one or more symptoms of a disorder.

The terms “drug” and “therapeutic agent” refer to a compound, or a pharmaceutical composition thereof, which is administered to a subject for treating, preventing, or ameliorating one or more symptoms of a disorder.

The term “disorder” as used herein is intended to be generally synonymous, and is used interchangeably with, the terms “disease,” “syndrome” and “condition” (as in medical condition), in that all reflect an abnormal condition (e.g., altered tissue energetics) of a subject's body or of one of its parts that impairs normal functioning and is typically manifested by distinguishing signs and symptoms.

In certain embodiments, the (−)-enantiomer of perhexyline is administered to the subject in an amount ranging from one of the following non-limiting ranges: 1 μg/kg to 100 mg/kg; 1 μg/kg to 100 mg/kg; 1 μg/kg to 10 mg/kg; 1 μg/kg to 1 mg/kg; 1 μg/kg to 100 μg/kg; 1 μg/kg to 10 μg/kg; 10 μg/kg to 100 mg/kg; 10 μg/kg to 10 mg/kg; 10 μg/kg to 1 mg/kg; 10 μg/kg to 100 μg/kg; 100 μg/kg to 100 mg/kg; 100 μg/kg to 10 mg/kg; 100 μg/kg to 1 mg/kg; 1 mg/kg to 10 mg/kg; and 10 mg/kg to 100 mg/kg body weight, and all subranges therebetween.

In certain embodiments, the (−)-enantiomer of perhexyline is administered to the subject in an amount to produce a plasma concentration of 2.4 mg/ml or less, 2.2 mg/ml or less, 2.0 mg/ml or less, 1.8 mg/ml or less, 1.6 mg/ml or less, 1.4 mg/ml or less, 1.2 mg/ml or less, 1.0 mg/ml or less, 0.8 mg/ml or less, 0.6 mg/ml or less, 0.4 mg/ml or less, 0.2 mg/ml or less, 0.1 mg/ml or less, or 0.05 mg/ml or less. In certain embodiments, the (−)-enantiomer of perhexyline is administered to the subject in an amount to produce a plasma concentration of 2.4 mg/ml or greater, 2.2 mg/ml or greater, 2.0 mg/ml or greater, 1.8 mg/ml or greater, 1.6 mg/ml or greater, 1.4 mg/ml or greater, 1.2 mg/ml or greater, 1.0 mg/ml or greater, 0.8 mg/ml or greater, 0.6 mg/ml or greater, 0.4 mg/ml or greater, 0.2 mg/ml or greater, 0.1 mg/ml or greater, or 0.05 mg/ml or greater.

In certain embodiments, the (−)-enantiomer of perhexyline is administered to the subject in an amount to produce a plasma concentration from one of the following non-limiting ranges: 0.01-0.6 mg/L; 0.025-0.6 mg/L; 0.05-0.6 mg/L; 0.075-0.6 mg/L; 0.1-0.6 mg/L; 0.125-0.6 mg/L; 0.15-0.6 mg/L; 0.2-0.6 mg/L; 0.3-0.6 mg/L; 0.4-0.6 mg/L; 0.5-0.6 mg/L; 0.01-0.5 mg/L; 0.025-0.5 mg/L; 0.05-0.5 mg/L; 0.075-0.5 mg/L; 0.1-0.5 mg/L; 0.125-0.5 mg/L; 0.15-0.5 mg/L; 0.2-0.5 mg/L; 0.3-0.5 mg/L; 0.4-0.5 mg/L; 0.01-0.4 mg/L; 0.025-0.4 mg/L; 0.05-0.4 mg/L; 0.075-0.4 mg/L; 0.1-0.4 mg/L; 0.125-0.4 mg/L; 0.15-0.4 mg/L; 0.2-0.4 mg/L; 0.3-0.4 mg/L; 0.01-0.3 mg/L; 0.025-0.3 mg/L; 0.05-0.3 mg/L; 0.075-0.3 mg/L; 0.1-0.3 mg/L; 0.125-0.3 mg/L; 0.15-0.3 mg/L; 0.2-0.3 mg/L; 0.01-0.2 mg/L; 0.025-0.2 mg/L; 0.05-0.2 mg/L; 0.075-0.2 mg/L; 0.1-0.2 mg/L; 0.125-0.2 mg/L; 0.15-0.2 mg/L; 0.01-0.1 mg/L; 0.025-0.1 mg/L; 0.05-0.1 mg/L; 0.075-0.1 mg/L; 0.01-0.075 mg/L; 0.025-0.075 mg/L; 0.05-0.075 mg/L; 0.01-0.05 mg/L; 0.025-0.05 mg/L; or 0.01-0.025 mg/L and all subranges therebetween.

In certain embodiments, the amount of (−)-perhexyline administered to the subject produces a plasma concentration of less than 0.6 mg/L. In certain embodiments, the amount of (−)-perhexyline administered to the subject produces a plasma concentration in one of the following ranges: 0.05-0.30 mg/L; 0.05-0.60 mg/L; 0.05-0.90 mg/L; 0.15-1.20 mg/L; 0.15-0.60 mg/L; 0.15-0.90 mg/L; and 0.15-1.20 mg/L and all subranges therebetween.

In certain embodiments, the amount of (−)-perhexyline administered to the subject comprises a loading dose of about 50 mg, about 100 mg, about 150 mg or about 200 mg. In certain embodiments, the amount of (−)-perhexyline administered to the subject comprises a loading dose of 50 mg or less, 100 mg or less, 150 mg or less, or 200 mg or less and all subranges therebetween. In certain embodiments, the amount of (−)-perhexyline administered to the subject comprises a loading dose of 50 mg or greater, 100 mg or greater, 150 mg or greater, or 200 mg or greater and all subranges therebetween.

In certain embodiments, the amount of (−)-perhexyline administered to the subject comprises a maintenance dose of about 50 mg, about 100 mg, about 150 mg or about 200 mg and all subranges therebetween. In certain embodiments, the amount of (−)-perhexyline administered to the subject comprises a maintenance dose of 50 mg or less, 100 mg or less, 150 mg or less, or 200 mg or less and all subranges therebetween. In certain embodiments, the amount of (−)-perhexyline administered to the subject comprises a maintenance dose of 50 mg or greater, 100 mg or greater, 150 mg or greater, or 200 mg or greater and all subranges therebetween. The maintenance dose may for example be administered daily, every second day, twice a week, once a week or once a fortnight and all subranges therebetween.

In certain embodiments, the amount of (−)-perhexyline administered to the subject comprises a loading dose as described herein in conjunction with a maintenance dose as described herein. It will be appreciated that the loading dose may be provided over a suitable time period, for example 5-7 days, and the maintenance dose may also be provided over a suitable time period. In certain embodiments, the maintenance dose may be provided over a period of 1 month, 2 months, 3 months, 6 months, 1 year or indefinitely.

In certain embodiments, the amount of (−)-perhexyline administered to the subject comprises 100-150 mg once daily for 5-7 days and 50 mg daily thereafter, or 50-100 mg once daily for 5-7 days and 50 mg daily thereafter. In certain embodiments, the amount of (−)-perhexyline administered to the subject comprises 50-75 mg once a week or 25-50 mg once a week. In certain embodiments, the amount of (−)-perhexyline administered to the subject comprises 50 mg daily on a continuous basis, with no loading dose. 100-150 mg once daily for 5-7 days and 50 mg daily thereafter, or 50-100 mg once daily for 5-7 days and 50-100 mg or greater daily thereafter.

The enantiomer of perhexyline may be administered to the subject in a suitable form. In this regard, the terms “administering” or “providing” includes administering an enantiomer of perhexyline, and/or administering a salt, prodrug or derivative of perhexyline, that will form an effective amount of the active agent within the body of the subject. The terms include routes of administration that are systemic (e.g., via injection such as intravenous injection, orally in a tablet, pill, capsule, or other dosage form useful for systemic administration of pharmaceuticals), and topical (e.g., creams, solutions, suppositories, sublingual and the like, including solutions such as mouthwashes, for topical oral administration). Methods of drug administration are generally known in the art.

The enantiomer of perhexyline may be administered alone or may be delivered in a mixture with other therapeutic agents and/or agents that enhance, stabilise or maintain the activity of the enantiomer of perhexyline.

In certain embodiments, the methods further comprise administering to the subject another active agent, such as one or more metabolic agents, or other agents such as one or more of an ACE inhibitor, a beta blocker, an aldosterone antagonist, a diuretic, a nitrate, a calcium channel blocker, glucose, insulin, potassium, an insulin sensitiser, and glucagon-like peptide-1 (GLP-1).

In certain embodiments, an administration vehicle (e.g., pill, tablet, implant, injectable solution, etc.) contains both the enantiomer of perhexyline and additional agent(s).

The methods of administration may also include combination therapy. In this regard, the subject is treated or given another drug or treatment modality in conjunction with the enantiomer of perhexyline as described herein. This combination therapy can be sequential therapy where the subject is treated first with one agent and then the other agent, or the two or more treatment modalities are given simultaneously.

“Co-administering” or “co-administration” refers to the administration of two or more therapeutic or active agents together at one time. The two or more therapeutic or active agents can be co-formulated into a single dosage form or “combined dosage unit”, or formulated separately and subsequently combined into a combined dosage unit, typically for intravenous administration or oral administration. Dosage units for other administration routes are contemplated.

When administered to a subject in need thereof, the effective dosage may vary depending upon the mode of administration, the condition, and severity thereof, as well as the various physical factors related to the subject being treated. As discussed herein, suitable daily doses range from about 1 μg/kg to about 20 mg/kg. The daily dosages are expected to vary with route of administration, and the nature of the enantiomer of perhexyline administered. In certain embodiments the methods comprise administering to the subject escalating doses of the enantiomer of perhexyline and/or repeated doses. In certain embodiments, the enantiomer of perhexyline is administered orally. In certain embodiments, the enantiomer of perhexyline is administered via injection, such as intravenous injection. In certain embodiments, the enantiomer of perhexyline is administered parenterally. In certain embodiments, the enantiomer of perhexyline is administered by direct introduction to the lungs, such as by aerosol administration, by nebulized administration, and by being instilled into the lung. In certain embodiments, the enantiomer of perhexyline is administered by implant. In certain embodiments, the enantiomer of perhexyline is administered by subcutaneous injection, intraarticularly, rectally, intranasally, intraocularly, vaginally, or transdermally. Other administration routes are contemplated.

Pharmaceutical compositions containing the perhexyline enantiomer described herein may be administered at once, or multiple times at intervals of time. It is understood that the precise dosage and duration of treatment may vary with the age, weight, and condition of the patient being treated, and may be determined empirically using known testing protocols or by extrapolation from in vivo or in vitro test or diagnostic data. It is further understood that for any particular individual, specific dosage regimens should be adjusted over time according to the individual need and the professional judgment of the person administering or supervising the administration of the formulations.

In the case wherein the patient's condition does not improve, upon the physician's discretion the administration of the compounds may be administered chronically, that is, for an extended period of time, including throughout the duration of the patient's life in order to ameliorate or otherwise control or limit the symptoms of the patient's disorder. In the case wherein the patient's status does improve, upon the doctor's discretion the administration of the compounds may be given continuously or temporarily suspended for a certain length of time (i.e., a “drug holiday”).

Once improvement of the patient's condition has occurred, a maintenance dose is administered if necessary. Subsequently, the dosage or the frequency of administration, or both, can be reduced, as a function of the symptoms, to a level at which the improved disease, disorder or condition is retained. Patients can, however, require intermittent treatment on a long-term basis upon any recurrence of symptoms.

“Intravenous administration” is the administration of substances directly into a vein. “Oral administration” is a route of administration where a substance is taken through the mouth, and includes buccal, sublabial and sublingual administration, as well as enteral administration and that through the respiratory tract, unless made through e.g. tubing so the medication is not in direct contact with any of the oral mucosa. Typical forms for the oral administration of therapeutic agents includes the use of tablets or capsules.

The term “release controlling excipient” refers to an excipient whose primary function is to modify the duration or place of release of the active substance from a dosage form as compared with a conventional immediate release dosage form.

The term “nonrelease controlling excipient” refers to an excipient whose primary function do not include modifying the duration or place of release of the active substance from a dosage form as compared with a conventional immediate release dosage form.

In certain embodiments, the enantiomer of perhexyline is administered as an immediate release formulation. The term “immediate release formulation” is a formulation which is designed to quickly release a therapeutic or active agent in the body over a shortened period of time.

In certain embodiments, the enantiomer of perhexyline is administered as a controlled release formulation, a modified release formulation, a sustained release formulation or an extended release formulation.

In certain embodiments, the enantiomer of perhexyline is administered as a sustained release formulation. The term “sustained release formulation” is a formulation which is designed to slowly release a therapeutic or active agent in the body over an extended period of time.

In certain embodiments, the enantiomer of perhexyline is administered as an extended release formulation.

The enantiomer of perhexyline may be formulated into a controlled release formulation, a modified release formulation, a sustained release formulation or an extended release formulation by a suitable method. For example, modified release formulations and extended release formulations are as described generally in U.S. Pat. No. 8,173,708, U.S. Pat. No. 4,606,909 and U.S. Pat. No. 4,769,027 (each of which are hereby incorporated by reference).

For example, the formulation may comprise a multiplicity of individually coated or microencapsulated units that are made available upon disintegration of the formulation (for example a pill or tablet) in the stomach of the subject. Each of the individually coated or microencapsulated units may contain cross-sectionally substantially homogenous cores containing particles of a sparingly soluble active substance, the cores being coated with a coating that is substantially resistant to gastric conditions but which is erodable under the conditions prevailing in the gastrointestinal tract.

Extended release formulations may also involve pills of pharmaceutically acceptable material (e.g., sugar/starch, salts, and waxes) coated with a water permeable polymeric matrix containing the enantiomer of perhexyline and next overcoated with a water-permeable film containing dispersed within it a water soluble particulate pore forming material.

Alternatively, the enantiomer of perhexyline may be prepared in a formulation using a multilayered controlled release pharmaceutical dosage form. The dosage form contains a plurality of coated particles wherein each has multiple layers about a core containing the enantiomer of perhexyline and whereby the core and at least one other layer of active is overcoated with a controlled release barrier layer, therefore providing at least two controlled releasing layers from the multilayered coated particle.

In certain embodiments, the method comprises determining whether the subject is not a poor metabolizer of perhexyline. Methods for determining whether a subject is not a poor metabolizer, or whether the subject is a poor metabolizer, are known in the art.

In certain embodiments, substantially enantiomerically pure (i.e., enantiopure) (−)-perhexyline (and/or a pharmaceutically acceptable salt, prodrug or derivative thereof) is used in a pharmaceutical composition or in the manufacture of a medicament. In certain embodiments, substantially enantiopure (−)-perhexyline (and/or a pharmaceutically acceptable salt, prodrug or derivative thereof) is used in a pharmaceutical composition and/or the preparation of a medicament for preventing and/or treating a disease, condition or state associated with impaired tissue energetics.

Certain embodiments of the present disclosure provide use of substantially enantiopure (−)-perhexyline and/or a pharmaceutically acceptable salt, prodrug or derivative thereof in the manufacture of a medicament for preventing and/or treating a disease, condition or state associated with impaired tissue energetics.

Certain embodiments of the present disclosure provide a pharmaceutical composition comprising substantially enantiopure (−)-perhexyline and/or a pharmaceutically acceptable salt, prodrug or derivative thereof.

In certain embodiments, the pharmaceutical composition or medicament comprises (−)-enantiomer at 90% or greater of the total perhexyline in the composition or medicament. In certain embodiments, the pharmaceutical composition or medicament comprises (−)-enantiomer at 95% or greater; 96% or greater; 97% or greater; 98% or greater; or 99% or greater of the total perhexyline in the composition or medicament, and all subranges therebetween.

In certain embodiments, the composition or medicament comprises 25 to 250 mg (−)-perhexyline, and all subranges therebetween. In certain embodiments, the composition or medicament comprises 25 mg (−)-perhexyline; 50 mg (−)-perhexyline; 75 mg (−)-perhexyline; 100 mg (−)-perhexyline; 125 mg (−)-perhexyline; 150 mg (−)-perhexyline; 175 mg (−)-perhexyline; 200 mg (−)-perhexyline; 225 mg (−)-perhexyline or 250 mg (−)-perhexyline; or an amount of (−)-perhexyline at about the aforementioned amounts. In certain embodiments, the composition or medicament comprises 25 mg or less (−)-perhexyline; 50 mg or less (−)-perhexyline; 75 mg or less (−)-perhexyline; 100 mg or less (−)-perhexyline; 125 mg or less (−)-perhexyline; 150 mg or less (−)-perhexyline; 175 mg or less (−)-perhexyline; 200 mg or less (−)-perhexyline or, 225 mg or less (−)-perhexyline or 250 mg or less (−)-perhexyline.

In certain embodiments, the composition or medicament comprises an effective amount of (−)-perhexyline that when administered to a subject in need thereof once daily produces a plasma concentration as described herein.

In certain embodiments, the composition or medicament comprises an effective amount of (−)-perhexyline that when administered to a subject in need thereof once daily produces a plasma concentration in one of the following ranges: 0.05-0.30 mg/L; 0.05-0.60 mg/L; 0.05-0.90 mg/L; 0.05-01.20 mg/L; 0.15-0.30 mg/L; 0.15-0.60 mg/L; 0.15-0.90 mg/L; 0.15-1.20 mg/L and all subranges therebetween.

In certain embodiments, the composition or medicament when administered to a subject in need thereof does not result in substantial hepatotoxicity and/or neuropathy. In certain embodiments, the composition or medicament when administered to a subject in need thereof results in reduced hepatoxicity and/or neuropathy as compared to administration of a composition or medicament comprising an equivalent amount of racemic perhexyline or the (+)-enantiomer.

It is also envisaged that a pharmaceutical composition comprising (−)-perhexyline will have an improved safety profile as compared to the racemate, that the dose of (−)-perhexyline will be able to be reduced as compared to the racemate, and that the composition will be safer to use in subjects suffering from, or susceptible to, fatty liver disease or steatohepatitis, than the racemate.

It is also envisaged that a pharmaceutical composition comprising (−)-perhexyline will be safer to use in subjects suffering from, or susceptible to, fatty liver disease, steatohepatitis and/or peripheral neuropathy, than the racemate.

Certain embodiments provide a method for preventing and/or treating a disease, condition or state associated with altered tissue energetics, the method comprising administering to a subject in need thereof a pharmaceutical composition or medicament as described herein.

In certain embodiments, the enantiomer of perhexyline is provided with a pharmaceutically acceptable carrier suitable for administering a pharmaceutical composition to a subject in need thereof.

Carriers may be chosen based on the route of administration as described herein, the location of the target issue, the form of the (−)-enantiomer of perhexyline being delivered, the time course of delivery of the drug, etc. The term “pharmaceutically acceptable carrier,” “pharmaceutically acceptable excipient,” “physiologically acceptable carrier,” or “physiologically acceptable excipient” refers to a pharmaceutically-acceptable material, composition, or vehicle, such as a liquid or solid filler, diluent, excipient, solvent, or encapsulating material. Non-limiting examples include a solid, semi-solid or liquid filler, diluent, encapsulating material or formulation auxiliary of any type that is substantially inert pharmacologically. An example of a pharmaceutically acceptable carrier is physiological saline. Other physiologically acceptable carriers and their formulations are known in the art. Some examples of materials which can serve as pharmaceutically acceptable carriers include, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil; safflower oil; sesame oil; olive oil; corn oil and soybean oil; glycols such as propylene glycol; esters such as ethyl oleate and ethyl laurate; agar; detergents such as TWEEN 80; buffering agents such as magnesium hydroxide and aluminium hydroxide; alginic acid; pyrogen-free water; isotonic saline; Ringer's solution; ethyl alcohol; and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present. Each a “pharmaceutically acceptable” material should be compatible with the other ingredients of a pharmaceutical formulation. It must also be suitable for use in contact with the tissue or organ of humans and animals without excessive toxicity, irritation, allergic response, immunogenecity, or other problems or complications, commensurate with a reasonable benefit/risk ratio. See, Remington: The Science and Practice of Pharmacy, 21st Edition; Lippincott Williams & Wilkins: Philadelphia, Pa., 2005; Handbook of Pharmaceutical Excipients, 5th Edition; Rowe et al, Eds., The Pharmaceutical Press and the American Pharmaceutical Association: 2005; and Handbook of Pharmaceutical Additives, 3rd Edition; Ash and Ash Eds., Gower Publishing Company: 2007; Pharmaceutical Preformulation and Formulation, Gibson Ed., CRC Press LLC: Boca Raton, Fla., 2004), each of which are incorporated by reference in their entirety.

In certain embodiments, the (−)-enantiomer of perhexyline may be administered or present in a pharmaceutical composition as a pharmaceutically acceptable salt, solvate or prodrug thereof. The term “pharmaceutically acceptable salt” refers to acid addition salts or metal complexes which are commonly used in the pharmaceutical industry. Metal complexes include zinc, iron, and the like. Suitable acids for use in the preparation of pharmaceutically acceptable salts include, but are not limited to, acetic acid, 2,2-dichloroacetic acid, acylated amino acids, adipic acid, alginic acid, ascorbic acid, L-aspartic acid, benzenesulfonic acid, benzoic acid, A-acetamidobenzoic acid, boric acid, (+)-camphoric acid, camphorsulfonic acid, (+)-(IS)-camphor-10-sulfonic acid, capric acid, caproic acid, caprylic acid, cinnamic acid, citric acid, cyclamic acid, cyclohexanesulfamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucuronic acid, L-glutamic acid, α-oxo-glutaric acid, glycolic acid, hippuric acid, hydrobromic acid, hydrochloric acid, hydroiodic acid, (+)-L-lactic acid, (±)-DL-lactic acid, lactobionic acid, lauric acid, maleic acid, (−)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, perchloric acid, phosphoric acid, L-pyroglutamic acid, saccharic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid, trifluoroacetic acid, undecylenic acid, suberic acid, valeric acid and the like.

Suitable bases for use in the preparation of pharmaceutically acceptable salts, including, but not limited to, inorganic bases, such as magnesium hydroxide, calcium hydroxide, potassium hydroxide, zinc hydroxide, or sodium hydroxide; and organic bases, such as primary, secondary, tertiary, and quaternary, aliphatic and aromatic amines, including L-arginine, benethamine, benzathine, choline, deanol, diethanolamine, diethylamine, dimethylamine, dipropylamine, diisopropylamine, 2-(diethylamino)-ethanol, ethanolamine, ethylamine, ethylenediamine, isopropylamine, N-methyl-glucamine, hydrabamine, IH-imidazole, L-lysine, morpholine, 4-(2-hydroxyethyl)-morpholine, methylamine, piperidine, piperazine, propylamine, pyrrolidine, I-(2-hydroxyethyl)-pyrrolidine, pyridine, quinuclidine, quinoline, isoquinoline, secondary amines, triethanolamine, trimethylamine, triethylamine, N-methyl-D-glucamine, 2-amino-2-(hydroxymethyl)-1,3-propanediol, tromethamine, and the like.

The active agent ((−)-perhexyline) as disclosed herein may also be designed as a prodrug, which is a functional derivative of the compound as disclosed herein and is readily convertible into the parent compound in vivo. Prodrugs are often useful because, in some situations, they may be easier to administer than the parent compound. They may, for instance, be bioavailable by oral administration whereas the parent compound is not. The prodrug may also have enhanced solubility in pharmaceutical compositions over the parent compound. A prodrug may be converted into the parent drug by various mechanisms, including enzymatic processes and metabolic hydrolysis. See Harper, Progress in Drug Research 1962, 4, 221-294; Morozowich et al. in “Design of Biopharmaceutical Properties through Prodrugs and Analogs,” Roche Ed., APHA Acad. Pharm. Sci. 1977; “Bioreversible Carriers in Drug in Drug Design, Theory and Application,” Roche Ed., APHA Acad. Pharm. Sci. 1987; “Design of Prodrugs,” Bundgaard, Elsevier, 1985; Wang et al., Curr. Pharm. Design 1999, 5, 265-287; Pauletti et al., Adv. Drug. Delivery Rev. 1997, 27, 235-256; Mizen et al., Pharm. Biotech. 1998, 11, 345-365; Gaignault et al., Pract. Med. Chem. 1996, 671-696; Asgharnejad in “Transport Processes in Pharmaceutical Systems,” Amidon et al., Ed., Marcell Dekker, 185-218, 2000; Balant et al., Eur. J. Drug Metab. Pharmacokinet. 1990, 15, 143-53; Balimane and Sinko, Adv. Drug Delivery Rev. 1999, 39, 183-209; Browne, Clin. Neuropharmacol. 1997, 20, 1-12; Bundgaard, Arch. Pharm. Chem. 1979, 86, 1-39; Bundgaard, Controlled Drug Delivery 1987, 17, 179-96; Bundgaard, Adv. Drug Delivery Rev. 1992, 8, 1-38; Fleisher et al, Adv. Drug Delivery Rev. 1996, 19, 115-130; Fleisher et al., Methods Enzymol. 1985, 112, 360-381; Farquhar et al., J. Pharm. Sci. 1983, 72, 324-325; Freeman et al., J. Chem. Soc, Chem. Commun. 1991, 875-877; Friis and Bundgaard, Eur. J. Pharm. Sci. 1996, 4, 49-59; Gangwar et al., Des. Biopharm. Prop. Prodrugs Analogs, 1977, 409-421; Nathwani and Wood, Drugs 1993, 45, 866-94; Sinhababu and Thakker, Adv. Drug Delivery Rev. 1996, 19, 241-273; Stella et al., Drugs 1985, 29, 455-73; Tan et al., Adv. Drug Delivery Rev. 1999, 39, 117-151; Taylor, A/v. Drug Delivery Rev. 1996, 19, 131-148; Valentino and Borchardt, Drug Discovery Today 1997, 2, 148-155; Wiebe and Knaus, Adv. Drug Delivery Rev. 1999, 39, 63-80; Waller et al., Br. J. Clin. Pharmac. 1989, 28, 497-507.

In certain embodiments, the pharmaceutical compositions or medicament comprises other therapeutic agents and/or agents that enhance, stabilise or maintain the activity of the active. Examples of other agents are as described herein.

Oral formulations containing the enantiomer of perhexyline as described herein may comprise any conventionally used oral forms, including tablets, capsules, buccal forms, troches, lozenges and oral liquids, suspensions or solutions. Capsules may contain mixtures of the active compound(s) with inert fillers and/or diluents such as the pharmaceutically acceptable starches (e.g. corn, potato or tapioca starch), sugars, artificial sweetening agents, powdered celluloses, such as crystalline and microcrystalline celluloses, flours, gelatins, gums, etc. Useful tablet formulations may be made by conventional compression, wet granulation or dry granulation methods and utilize pharmaceutically acceptable diluents, binding agents, lubricants, disintegrants, surface modifying agents (including surfactants), suspending or stabilizing agents, including magnesium stearate, stearic acid, talc, sodium lauryl sulfate, microcrystalline cellulose, carboxymethylcellulose calcium, polyvinylpyrrolidone, gelatin, alginic acid, acacia gum, xanthan gum, sodium citrate, complex silicates, calcium carbonate, glycine, dextrin, sucrose, sorbitol, dicalcium phosphate, calcium sulfate, lactose, kaolin, mannitol, sodium chloride, talc, dry starches and powdered sugar. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidol silicon dioxide, phosphates, sodium dodecylsulfate, magnesium aluminium silicate, and triethanolamine. Oral formulations may utilize standard delay or time-release formulations to alter the absorption of the enantiomer of perhexyline. The oral formulation may also consist of administering the active ingredient in water or a fruit juice, containing appropriate solubilizers or emulsifiers as needed.

In certain embodiments, an oral formulation of perhexyline may contain one or more of lactose, maize starch, sucrose and purified talc.

In certain embodiments, it may be desirable to administer the enantiomer of perhexyline directly to the airways in the form of an aerosol. Formulations for the administration of aerosol forms are known in the art.

In certain embodiments, the enantiomer of perhexyline may also be administered parenterally (such as directly into the joint space) or intraperitoneally. For example, solutions or suspensions of these compounds in a non-ionised form or as a pharmacologically acceptable salt can be prepared in water suitably mixed with a surfactant such as hydroxy-propylcellulose. Dispersions can also be prepared in glycerol, liquid polyethylene glycols and mixtures thereof in oils.

In certain embodiments, the enantiomer of perhexyline may also be administered by injection. Pharmaceutical forms suitable for injectable use include sterile aqueous solutions or dispersions and sterile powders for the extemporaneous preparation of sterile injectable solutions or dispersions. The carrier can be a solvent or dispersion medium containing, for example, water, ethanol, polyol (e.g., glycerol, propylene glycol and liquid polyethylene glycol), suitable mixtures thereof, and vegetable oils.

In certain embodiments, the enantiomer of perhexyline may also be administered intravenously. Compositions containing the enantiomer of perhexyline described herein suitable for intravenous administration may be formulated by a skilled person.

In certain embodiments, the enantiomer of perhexyline may also be administered transdermally. Transdermal administrations are understood to include all administrations across the surface of the body and the inner linings of bodily passages including epithelial and mucosal tissues. Such administrations may be carried out using the modulator as described herein, or pharmaceutically acceptable salts thereof, in lotions, creams, foams, patches, suspensions, solutions, and suppositories (rectal and vaginal).

Transdermal administration may also be accomplished through the use of a transdermal patch containing the active compound and a carrier that is inert to the active compound, is non toxic to the skin, and allows delivery of the agent for systemic absorption into the blood stream via the skin. The carrier may take any number of forms such as creams and ointments, pastes, gels, and occlusive devices. The creams and ointments may be viscous liquid or semisolid emulsions of either the oil-in-water or water-in-oil type. Pastes comprised of absorptive powders dispersed in paraffin containing the active ingredient may also be suitable. A variety of occlusive devices may be used to release the active ingredient into the blood stream such as a semi-permeable membrane covering a reservoir containing the active ingredient with or without a carrier, or a matrix containing the active ingredient.

In certain embodiments, the enantiomer of perhexyline may also be administered by way of a suppository. Suppository formulations may be made from traditional materials, including cocoa butter, with or without the addition of waxes to alter the suppository's melting point, and glycerin. Water soluble suppository bases, such as polyethylene glycols of various molecular weights, may also be used.

Additional numerous various excipients, dosage forms, dispersing agents and the like that are suitable for use in connection with the administration of the enantiomer of perhexyline and/or the formulation into medicaments or pharmaceutical compositions. See Remington's Pharmaceutical Sciences, supra.

In certain embodiments, enantiopure perhexyline (and/or a pharmaceutically acceptable salt, prodrug or derivative thereof) is administered to prevent and/or treat a disease, condition or state associated with impaired cardiac tissue energetics.

Certain embodiments of present disclosure provide a method for preventing and/or treating a disease, condition or state associated with impaired cardiac tissue energetics in a subject in need thereof, the method comprising administering to the subject a therapeutically effective amount of substantially enantiopure (−)-perhexyline and/or a pharmaceutically acceptable salt, prodrug or derivative thereof.

In certain embodiments, the enantiopure (−)-perhexyline (and/or a pharmaceutically acceptable salt, prodrug or derivative thereof) is used in the in the manufacture of a medicament for preventing and/or treating a disease, condition or state associated with impaired cardiac tissue energetics in a subject in need thereof.

Certain embodiments of the present disclosure provide use of substantially enantiopure (−)-perhexyline, and/or a pharmaceutically acceptable salt, prodrug or derivative thereof in the manufacture of a medicament for preventing and/or treating a disease, condition or state associated with impaired cardiac tissue energetics in a subject in need thereof.

Certain embodiments of the present disclosure provide a method for preventing and/or treating in a subject in need thereof one of more of ischaemic heart disease, heart failure including systolic and diastolic heart failure, angina, refractory angina, ventricular hypertrophy, cardiomyopathy including congestive cardiomyopathy and hypertrophic cardiomyopathy, the method administering to the subject a therapeutically effective amount of substantially enantiopure (−)-perhexyline and/or a pharmaceutically acceptable salt, prodrug or derivative thereof.

Certain embodiments of the present disclosure provide use of substantially enantiopure (−)-perhexyline, and/or a pharmaceutically acceptable salt, prodrug or derivative thereof in the manufacture of a medicament for preventing and/or treating in a subject in need thereof one of more of ischaemic heart disease, heart failure including systolic and diastolic heart failure, angina, refractory angina, ventricular hypertrophy, cardiomyopathy including congestive cardiomyopathy and hypertrophic cardiomyopathy.

Certain embodiments of the present disclosure provide a method for preventing and/or treating type I or type II diabetes in a subject in need thereof, the method administering to the subject a therapeutically effective amount of substantially enantiopure (−)-perhexyline and/or a pharmaceutically acceptable salt, prodrug or derivative thereof.

Certain embodiments of the present disclosure provide use of substantially enantiopure (−)-perhexyline, and/or a pharmaceutically acceptable salt, prodrug or derivative thereof in the manufacture of a medicament for preventing and/or treating type I or type II diabetes in a subject in need thereof.

In certain embodiments, administration of the enantiopure (−)-perhexyline reduces adverse effects in the subject.

Certain embodiments of the present disclosure provide a method for administration of perhexyline with reduced adverse effects, as compared to administration of the racemate.

Certain embodiments of the present disclosure provide a method for administration of perhexyline with reduced adverse effects, the method comprising administering to a subject in need thereof a therapeutically effective amount of substantially enantiopure (−)-perhexyline and/or a pharmaceutically acceptable salt, prodrug or derivative thereof.

Examples of subjects are as described herein. In certain embodiments, the subject is susceptible to or suffering from a disease, condition or state associated with altered tissue energetics. In certain embodiments, the subject is susceptible to or suffering from a disease, condition or state associated with impaired tissue energetics. Examples of a disease, condition or state associated with altered tissue energetics are as described herein. In certain embodiments, the disease, condition or state is associated with impaired cardiac tissue energetics. In certain embodiments, the disease, condition or state comprises one or more of ischaemic heart disease, heart failure including systolic and diastolic heart failure, angina, refractory angina, ventricular hypertrophy, cardiomyopathy, including congestive cardiomyopathy and hypertrophic cardiomyopathy.

In certain embodiments, the disease, condition or state is associated with altered hepatic tissue energetics.

In certain embodiments, the subject has one or more of the following characteristics: ischaemia, increased tissue NADH/NAD+, reduced tissue pyruvate dehydrogenase activity, increased anaerobic glycolysis, increased fatty acid β-oxidation, reduced phosphocreatine concentration, reduced oxidative phosphorylation, increased insulin resistance, and a reduced ratio of phosphocreatine to ATP.

In certain embodiments, the method for administration of perhexyline comprises reducing one or more adverse effects in the subject as compared to administration of racemic perhexyline. Examples of adverse effects are as described herein. In certain embodiments, the one or more adverse effects comprise hepatotoxicity and/or peripheral neuropathy.

In certain embodiments, the administration of (−)-perhexyline increases hepatic glycogen content without substantially increasing hepatic lipid content and/or non-hepatic tissue lipid content in the subject.

In certain embodiments, the administration of (−)-perhexyline produces one or more of the following in the subject: increased glucose utilization, increased myocardial lactate utilization, reduced myocardial lactate accumulation, reduced long chain fatty acid utilization, and increased cardiac efficiency.

In certain embodiments, the amount of (−)-perhexyline administered to the subject produces a plasma concentration as described herein.

In certain embodiments, the amount of (−)-perhexyline administered to the subject produces a plasma concentration in one of the following ranges: 0.05-0.30 mg/L; 0.05-0.60 mg/L; 0.05-0.90 mg/L; 0.05-01.20 mg/L; 0.15-0.30 mg/L; 0.15-0.60 mg/L; 0.15-0.90 mg/L; 0.15-1.20 mg/L and all subranges therebetween.

Certain embodiments of the present disclosure provide a method for reducing one or more adverse effects in a subject in need thereof associated with administration of perhexyline, the method comprising administering to the subject a therapeutically effective amount of substantially enantiopure (−)-perhexyline and/or a pharmaceutically acceptable salt, prodrug or derivative thereof.

Certain embodiments of the present disclosure provide use of substantially enantiopure (−)-perhexyline and/or a pharmaceutically acceptable salt, prodrug or derivative thereof in the manufacture of a medicament for reducing one or more adverse effects in a subject in need thereof associated with perhexyline administration.

In certain embodiments, a product comprising an enantiomer of perhexyline is provided.

Certain embodiments of the present disclosure provide a combination product comprising an enantiomer of perhexyline; and instructions for administering the enantiomer of perhexyline to a subject in need thereof to prevent and/or treat one or more of the diseases, conditions or states as described herein.

Certain embodiments of the present disclosure provide a kit or article of manufacture for performing the methods as described herein is provided. The kit may comprise one or more modulators, agents, reagents, components, compositions, formulations, products and instructions as described herein. The kit or article of manufacture can include a container (such as a bottle) with a desired amount of at least one enantiomer of perhexyline (or pharmaceutical composition thereof) as disclosed herein. Further, such a kit or article of manufacture can further include instructions for use. The instructions can be attached to the container, or can be included in a package (such as a box or a plastic or foil bag) holding the container.

Certain embodiments of the present disclosure provide a kit for preventing and/or treating a disease, condition or state associated with altered tissue engergetics, the kit comprising an (−)-enantiomer of perhexyline and optionally comprising one or more of instructions for administering the enantiomer of perhexyline to a subject in need thereof.

Certain embodiments of the present disclosure provide methods for screening for new therapeutic agents.

In certain embodiments, the new therapeutic agents are candidate agents for preventing and/or treating a disease, condition or state as described herein. In certain embodiments, the new therapeutic agents are candidate metabolic agents. In certain embodiments, the new therapeutic agents are candidate cardiac metabolic agents.

Certain embodiments of the present disclosure provide a method for screening for an agent for preventing and/or treating a disease, condition or state associated with altered tissue energetics, the method comprising:
selecting a modified form of (−)-perhexyline; and
identifying the modified form of (−)-perhexyline as an agent for preventing and/or treating a disease, condition or state associated with altered tissue energetics.

Methods for identifying an agent for preventing and/or treating a disease, condition or state associated with altered tissue energetic are as described herein.

Diseases, conditions or states associated with altered tissue energetics are as described herein. In certain embodiments, the disease, condition or state associated with altered tissue energetics comprises a disease, condition or state associated with impaired tissue energetics. In certain embodiments, the disease, condition or state associated with altered tissue energetics comprises a disease, condition or state associated with impaired cardiac tissue energetics.

In certain embodiments, the methods for screening comprise administering the candidate agent to an animal or human subject and testing the effect of the candidate agent.

In certain embodiments, the methods for screening comprise administering the candidate agent to an animal or human subject and testing the effect of the candidate agent on hepatoxicity and/or neuropathy. In certain embodiments, the methods for screening comprise identifying the agent as an agent with reduced hepatoxicity and/or neuropathy. Methods for determining the extent of hepatotoxicity and/or neuropathy caused by a candidate agent are known in the art and are also as described herein.

In certain embodiments, the methods for screening comprise administering the candidate agent to an animal or human subject and testing the effect of the candidate agent as a metabolic agent. In certain embodiments, the methods for screening comprise identifying the agent as a metabolic agent. In certain embodiments, the metabolic agent is a cardiac metabolic agent.

In certain embodiments, the methods for screening comprise administering the agent to an animal or human subject and testing the effect of the agent to improve tissue energetics. In certain embodiments, the methods for screening comprise identifying the agent as an agent that promotes tissue energetics.

In certain embodiments, the methods for screening comprise administering the candidate agent to an animal or human and testing the effect of the candidate agent to improve carbohydrate utilization. In certain embodiments, the methods for screening comprise administering the candidate agent to an animal or human and testing the effect of the candidate agent to improve cardiac carbohydrate utilization.

In certain embodiments, the methods for screening comprise administering the agent to an animal or human subject and testing the effect of the agent to increase hepatic glycolysis or carbohydrate metabolism without substantially increasing hepatic fatty acid metabolism. In certain embodiments, the methods for screening comprise identifying the agent as agent that increases hepatic glycolysis or carbohydrate metabolism without substantially increasing hepatic fatty acid metabolism. Methods for identifying agent that increase hepatic glycolysis or carbohydrate metabolism without substantially increasing hepatic fatty acid metabolism are known in the art.

Certain embodiments of the present disclosure provide a method for screening for a cardiac metabolic agent.

Methods for screening agents for their activity as a cardiac metabolic agent are known in the art. For example, animal models of heart failure/cardiomyopathy include a Syrian Hamster cardiomyopathy model, the Pfeffer model, which employs coronary artery ligation in rats, isoprenaline-induced heart failure in rats; aortic banding in rats; and forms of genetically modified mouse strains. The effect of agents can be investigated in such animal models.

Certain embodiments of the present disclosure provide a method for screening for a metabolic agent for preventing and/or treating a disease, condition or state associated with altered or impaired cardiac tissue energetics, the method comprising:
selecting a modified form of (−)-perhexyline; and
identifying the modified form of (−)-perhexyline as a metabolic agent for preventing and/or treating a disease, condition or state associated with altered or impaired cardiac tissue energetics.

Certain embodiments of the present disclosure provide a method for screening for a cardiac metabolic agent with reduced hepatotoxicity and/or reduced neuropathy. Methods for identifying an agent with reduced hepatotoxicity and/or reduced neuropathy are known in the art.

Certain embodiments of the present disclosure provide a method for screening for a cardiac metabolic agent with reduced hepatotoxicity and/or reduced neuropathy, the method comprising:
selecting a modified form of (−)-perhexyline; and
identifying the modified form of (−)-perhexyline as an agent that increases hepatic carbohydrate metabolism without substantially increasing hepatic fatty acid metabolism, and
identifying the modified form of (−)-perhexyline as a cardiac metabolic agent with reduced hepatotoxicity and/or reduced neuropathy.

In certain embodiments, the method comprises administering the modified form of perhexyline to an animal or human subject and testing its ability as a cardiac metabolic agent. Methods for administration of perhexyline are as described herein.

Certain embodiments of the present disclosure provide a treatment regime with reduced adverse effects for treating a disease, condition or state associated with altered tissue energetics.

Certain embodiments of the present disclosure provide a treatment regime with reduced adverse effects for treating a disease, condition or state associated with altered tissue energetics, the treatment regime comprising:
administering to a subject in need thereof an effective amount of (−)-enantiomer of perhexyline and/or a pharmaceutically acceptable salt, prodrug or derivative thereof, substantially free of the other enantiomer; and
optionally administering one or more other compounds for treating the disease, condition or state.

Certain embodiments of the present disclosure provide a method for identifying a subject suitable for treatment with an (−)-enantiomer of perhexyline. In certain embodiments, the enantiomer of perhexyline is the (−)-enantiomer.

Certain embodiments of the present disclosure provide a method for identifying a subject suitable for treatment with (−)-perhexyline, the method comprising identifying a subject with one or more of the following characteristics: ischaemic heart disease, heart failure including systolic and diastolic heart failure, angina, refractory angina, ventricular hypertrophy, cardiomyopathy including congestive cardiomyopathy and hypertrophic cardiomyopathy, type I or type II diabetes, increased tissue NADH/NAD+, reduced tissue pyruvate dehydrogenase activity, increased anaerobic glycolysis, increased fatty acid β-oxidation, reduced phosphocreatine concentration, reduced oxidative phosphorylation, increased insulin resistance, and a reduced ratio of phosphocreatine to ATP.

Certain embodiments of the present disclosure provide a method for optimizing therapeutic efficacy of an (−)-enantiomer of perhexyline. In certain embodiments, the method for optimizing therapeutic efficacy comprises administering the enantiomer of perhexyline to a subject in need thereof. Administration of perhexyline to a subject in need thereof is as described herein.

In certain embodiments, the method for optimizing therapeutic efficacy comprises administering the (−)-enantiomer of perhexyline to a subject, determining a level of the enantiomer of perhexyline in the subject and altering the amount of the enantiomer of perhexyline subsequently administered to the subject.

Certain embodiments of the present disclosure provide a method for optimizing therapeutic efficacy of (−)-perhexyline, comprising:
administering (−)-perhexyline to a subject in need thereof;
determining a level of the (−)-perhexyline in the subject that is less than a first predetermined level corresponding to a second predetermined amount; and
increasing the amount of (−)-perhexyline subsequently administered to the subject.

Certain embodiments of the present disclosure provide a method for optimizing therapeutic efficacy of (−)-perhexyline, comprising:
administering (−)-perhexyline to a subject in need thereof;
determining a level of the (−)-perhexyline in the subject that is greater than a first predetermined level corresponding to a second predetermined amount; and
decreasing the amount of (−)-perhexyline subsequently administered to the subject.

The features of certain exemplary embodiments are illustrated by some of the following examples. It is to be understood that the following description is for the purpose of describing particular embodiments only and is not intended to be limiting with respect to the above description.

### Example 1

### In Vivo Myocardial Protection

Perhexyline maleate (racemate) was obtained from Sigma Pharmaceuticals. Pure (+)- and (−)-perhexyline were prepared as the maleate salt using the method described in Davies B J, Herbert M K, Culbert J A, Pyke S M, Coller J K, Somogyi A A, et al. (2006) Journal of chromatography. B, Analytical technologies in the biomedical and life sciences 832(1): 114-120.

The effect of racemic perhexyline, (+)-perhexyline and (−)-perhexyline on myocardial damage in a rat isoprenaline model of heart failure, using Troponin T as an indirect measure of myocardial damage was investigated. The use of a rat isoprenaline model of heart failure is as described in Teerlink, J R et al. (1994) “Progressive ventricular remodeling in response to diffuse isoproterenol-induced myocardial necrosis in rats” Circ Res, 75: 105-13.

DA rats were treated for 2 weeks with vehicle or 200 mg/kg/day of racemic, (+)- or (−)-perhexyline maleate, on day 15 myocardial necrosis was induced by i.p. injection of 50 mg/kg isoprenaline, with a second group of controls receiving an i.p. injection of saline (n=5). Cardiac troponin T levels were measured in plasma collected 3 hr following treatment by venipuncture of the tail vein.

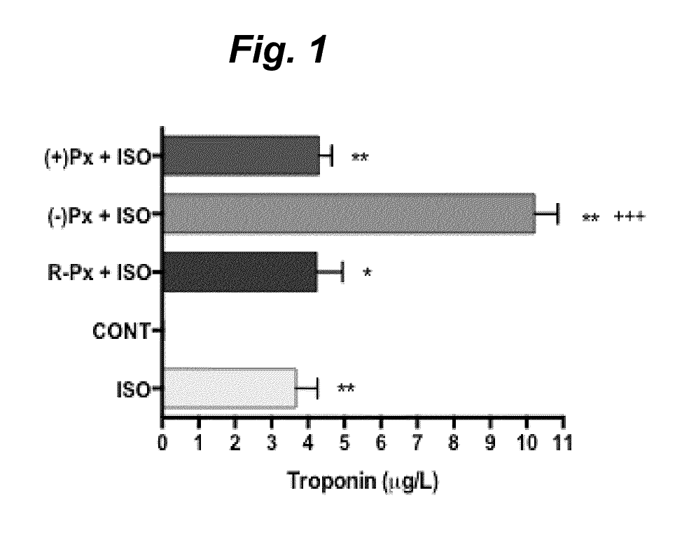

The data is shown in FIG. 1. Isoprenaline treatment caused a significant rise in troponins, which was not affected by pre-treatment with either racemic- or (+)-perhexyline, but was unexpectedly exacerbated by pre-treatment with (−)-perhexyline (1-way ANOVA, data shown as mean±sem).

This data suggested that (−)-perhexyline has an unexpected negative effect on myocardial damage which is not present on administration of the racemate or in the (+)-enantiomer. However, this data may also be explained by the possibility that the (−)-enantiomer and the (+)-enantiomer have different kinetics of Topinin T release. The kinetics of Tropinin T release were not measured in the experiment and it is possible that administration of (−)-perhexyline resulted in a faster release of Tropinin T (as measured at the 3 hr time point) than is observed with administration of the (+)-enantiomer.

### Example 2

### Enantioselectivity of Perhexyline on Hepatic Glucose and Fatty Acid Utilization

The severe clinical hepatotoxicity and neurotoxicity (peripheral neuropathy) associated with racemic-perhexyline is characterised by progressive development of steatosis, lysosomal lesions and phospholipidosis. Severe and prolonged inhibition of hepatic mitochondrial β-oxidation, and the subsequent increased esterification of fatty acids into triglycerides, can lead to microvesicular steatosis, and is a well established mechanism of drug-induced hepatotoxicity.

We therefore investigated the effects of each enantiomer on hepatic glucose and fatty acid utilization.

Adult female Dark Agouti (DA) rats (n=4 in each group) were administered 200 mg/kg daily of vehicle, racemic-, (+) or (−)-perhexyline maleate for a period of 2 months. The perhexyline maleate compound was administered mixed with peanut paste and coated onto standard rat chow. On day 56 of dosing, animals were anesthetized and blood was collected via a cardiac puncture in order to determine the concentrations of perhexyline enantiomers. Animals were then euthanized and hepatic, cardiac and neuronal tissues were harvested in order to determine perhexyline enantiomer and metabolite tissue concentrations and morphological changes. Tissues were cut in half and either immediately snap frozen in liquid nitrogen or placed into fixative solution for electron microscopy analysis.

Tissue was dissected into cubes of approximately 0.5 mm in each dimension and was fixed for one hour in electron microscopy (EM) fixative (4% formaldehyde and 1.5% glutaraldehyde in sodium cacodylate buffer, pH 7.2). The fixed tissue was post-fixed in 2% osmium tetroxide in sodium cacodylate buffer, en bloc stained with 2% uranyl acetate and dehydrated through 70%, 90% and 100% ethanol. Then, the tissue was processed through 1,2-epoxypropane, a 50/50 mixture of 1,2-epoxypropane and Procure 812 resin (Electron Microscopy Sciences, Fort Washington, USA) and two changes of 100% resin. Tissue and resin were transferred to Beem capsules and placed in an oven overnight at 90° C. Survey sections of tissue blocks were cut with glass knives and stained with Toluidine Blue. Thin sections were cut at approximately 100 nm thickness on a Porter-Blum ultramicrotome (Sorvall, Newtown, USA) using a diamond knife (Micro Star Technologies, Huntsville, USA). Thin sections were stained with Reynolds' lead citrate and examined in a Hitachi H-600 transmission electron microscope (Tokyo, Japan 1983). Glycogen and lipids were identified and total content was measured as a percentage of the field of view.

At 56 days, neuronal function was measured prior to euthanasia using von Frey filament testing. The thickness of each filament corresponds to a specific amount of pressure (in grams). Each animal was placed in a specifically designed plastic container, with the bottom replaced by mesh flooring. The animal was left for 10-15 minutes to settle into its new environment. In ascending order, von Frey filaments were applied from beneath the mesh flooring to the plantar surface of the paw until the filament buckled and was held there for a total of 10 seconds. A positive response was noted if the paw was withdrawn within the 10-second period. If a filament induced 6 positive responses, the grams corresponding to that filament were recorded as the paw withdrawal threshold.

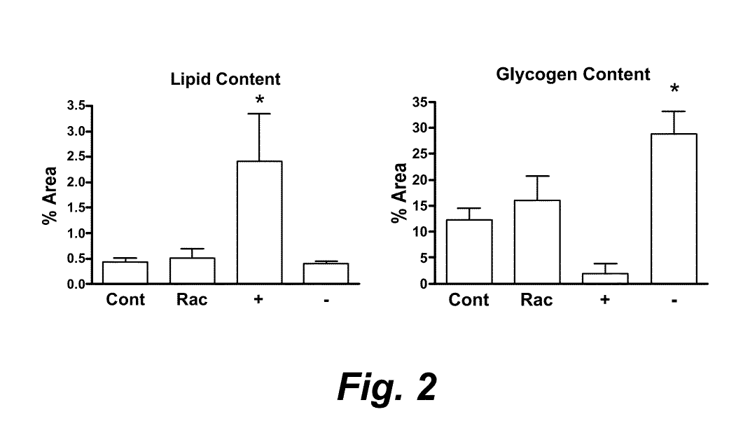

FIG. 2 shows histological assessment of mean (sd) hepatic lipid and glycogen content (% field area) in DA rats (n=4) treated with vehicle (Cont), racemic (Rac), (+)- or (−)-perhexyline for 8 weeks (*p<0.05 vs Cont).

The data presented in FIG. 2 shows that in Dark Agouti (DA) rats, (−)-perhexyline (at plasma concentrations of 0.22-0.39 mg/L) significantly increased hepatic glycogen but had no effect on lipids, whilst (+)-perhexyline (at plasma concentrations of 0.52-0.80 mg/L) significantly increased hepatic lipids and also appeared to decrease glycogen (FIG. 1).

The data demonstrates significant enantioselectivity with respect to perhexyline's in vivo effects on hepatic glucose and fatty acid utilization. The data presented clearly shows that only (+)-perhexyline causes hepatic steatosis, at plasma concentrations similar to those causing hepatotoxicity in humans.

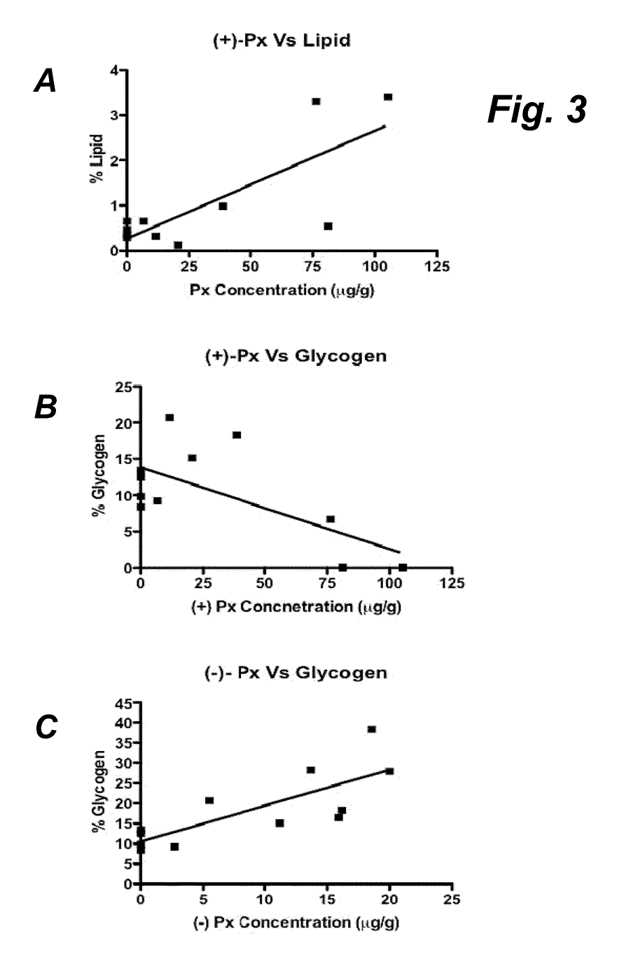

In order to test the hypothesis that the enantiomers exert contrasting effects on lipid and glycogen accumulation, correlations between hepatic enantiomer concentrations and effects were sought (including baseline data from controls and racemate-treated animals). As shown in FIG. 3A, there was a direct correlation (r=0.79, p=0.004) between hepatic concentration of (+)-enantiomer and lipid content, while a direct correlation was also seen (r=0.78, p=0.003) between (−)-enantiomer concentration and glycogen content (FIG. 3B). Furthermore, (+)-enantiomer concentrations were inversely correlated (r=−0.66, p=0.03) with glycogen content (FIG. 3C).

Stepwise multiple regression confirmed diverging effects of both (+)- and (−)-perhexyline concentrations on hepatic glycogen contents (p=0.02 for (+)-perhexyline, p=0.0004 for (−)-perhexyline, adjusted R2=0.76), but only a significant effect of (+)-perhexyline on hepatic lipid contents (p=0.0004, adjusted R2=0.62).

These results also show that (−)-perhexyline directly affects the pathways of carbohydrate utilization, perhaps partly explaining the greater antianginal efficacy of perhexyline compared to other inhibitors of β-oxidation. The accumulation of hepatic glycogen in rats treated with (−)-perhexyline is consistent with increased insulin sensitivity or an insulin-like effect. Such an effect would also be clinically beneficial in the myocardium, providing a mechanism for improved myocardial efficiency.

### Example 3

### Enantiomer's Contribution to Hepatic and Neural Toxicity

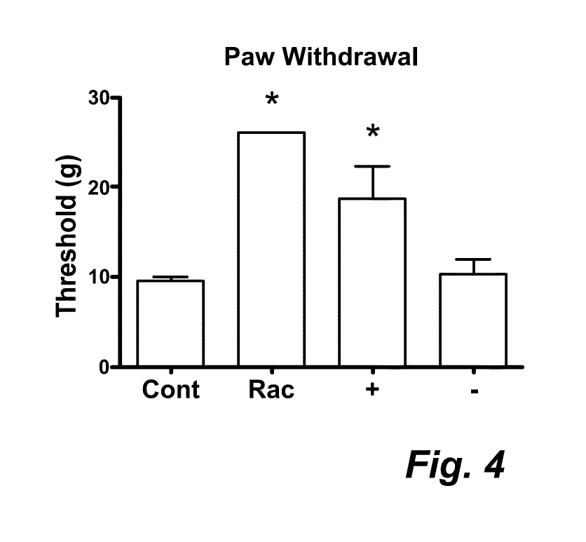

The data presented in FIG. 2 clearly shows that only (+)-perhexyline causes hepatic steatosis, at plasma concentrations similar to those causing hepatotoxicity in humans. To test the effect of each enantiomer on peripheral neural function, a Von Frey hairs model was used. FIG. 4 shows the mean (sd) paw withdrawal thresholds (g) in DA rats (n=4) treated for 8 weeks with vehicle (Cont), racemic (Rac), (+)- or (−)-perhexyline (*p<0.05 vs Cont).

As described, the data presented in FIG. 2 clearly show that only (+)-perhexyline causes hepatic steatosis, at plasma concentrations similar to those causing hepatotoxicity in humans. In the same study, testing of peripheral neural function using Von Frey hairs demonstrated that (+)-perhexyline caused a significant decline in neural sensory function, whereas (−)-perhexyline did not (FIG. 4).

One possible explanation is that both the major toxicities of the racemic formulation are due to inhibition of hepatic CPT-1 by (+)-perhexyline, highlighting the significant difference in pharmacological activities between the enantiomers.

In this regard, different CPT1 isoforms are expressed in liver (CPT1-A), adult cardiomyocytes (predominantly CPT1-B), and the central nervous system (CPT1-C), so it may be possible to dissociate perhexyline's beneficial effects on CPT1-B in the myocardium from its adverse effects in liver and nervous system. Consistent with this hypothesis, the inhibition of liver and heart CTP1 by racemic perhexyline displays atypical kinetics, which could reflect different inhibition affinities for the two CPT1 isoforms by the (+)- and (−)-enantiomers in the racemic mixture.

As well as inhibiting CPT1, perhexyline, like other weakly basic amphiphilic drugs with high lipophilicity, can become ionised and concentrated within mitochondria and lysosomes. At high concentrations, the trapping of protonated perhexyline within mitochondria uncouples oxidative phosphorylation, leading to a marked decrease in ATP synthesis and cell viability. Therefore in addition to its effects on CPT1, part of perhexyline's clinical toxicity may also be due to inhibition of oxidative phosphorylation at very high concentrations, a process that is unlikely to be enantioselective as it reflects the chemical amphiphilic and lipophilic nature of both enantiomers. Thus, an enantiomerically pure preparation of (−)-perhexyline may also reduce the clinical potential for toxicity by allowing the use of a lower overall dose of perhexyline.

As such, (−)-perhexyline is a candidate as a new myocardial metabolic agent, which may be devoid of the major adverse effects of the current racemic formulation and can be used as the basis for developing new structural analogues.

### Example 4

### Effects of Perhexyline on NOX2-Mediated Oxidative Stress, Inflammation and Nitric Oxide Responsiveness

Oxidative stress and inflammation are significant contributors to cardiovascular disease. In addition to changes in energy metabolism, the pathogenesis of acute coronary syndromes also involves inflammation and activation of immune cells, with superoxide formation contributing to plaque formation and rupture, enhanced nitric oxide clearance, and inhibition of platelet guanylate cyclase. Patients with ischaemic heart disease display changes in vascular structure and endothelial function, including decreased platelet and vascular responsiveness to nitric oxide, contributing to a shift in cardiovascular homeostasis towards vasoconstriction and thrombogenesis. In patients with high-risk acute coronary syndromes, impaired responsiveness of platelets to nitric oxide is an independent predictor of mortality and cardiovascular morbidity. Oxidative stress, decreased nitric oxide availability and altered nitric oxide responsiveness are also a feature of congestive heart failure, diabetes and aortic stenosis. The NOX family are a major source of cellular reactive oxygen species, and are not only expressed in neutrophils (NOX2) but also within the cardiovascular system, where NOX2 is expressed in endothelium, vascular smooth muscle, adventitial fibroblasts and cardiomyocytes, and has been implicated in cardiac hypertrophy, post-infarction remodelling and heart failure.

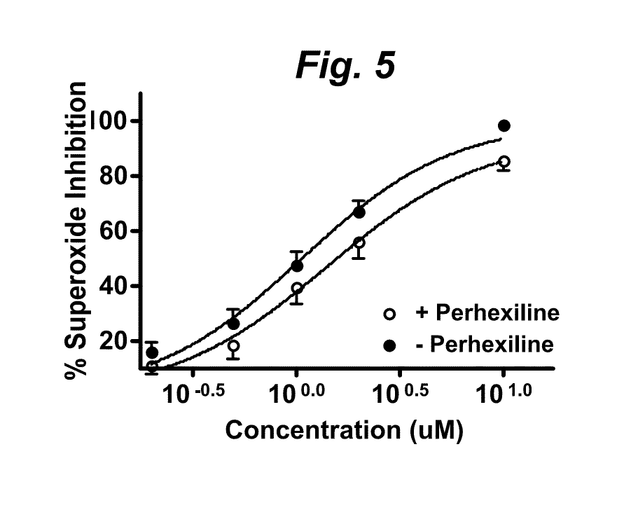

As an index of anti-inflammatory and nitric oxide-sparing potential, the relative potency of the enantiomers as inhibitors of ex vivo superoxide formation by NOX2 was investigated in human neutrophils. FIG. 5 shows the concentration-dependent inhibition of NOX2 by (+)- and (−)-perhexyline in neutrophils, as determined by measurement of percentage inhibition of superoxide formation.

Using neutrophils from 11 healthy young volunteers, both (+)- and (−)-perhexyline inhibited superoxide formation in a concentration-dependent manner (FIG. 3) with mean EC₅₀ values of 1.8 and 1.3 μM (p<0.05), respectively. Whilst this enantioselectivity is consistent with different therapeutic indices for the enantiomers, it can be seen that both still contribute to perhexyline's anti-inflammatory effects.

This data shows that while (−)-perhexyline has reduced clinical toxicity as compared to the racemate and the (+) enantiomer, both enantiomers have the same ability to inhibit NOX2-mediated superoxide formation, being an index of anti-inflammatory and nitric oxide-sparing potential.

This data also suggests that some of perhexyline's beneficial effects in heart failure and cardiomyopathy may involve anti-inflammatory activity that is independent of CPT1 inhibition.

### Example 5

### In Vitro CPT 1 Inhibition

Using liver and heart homogenates and purified mitochondrial fractions, it was found that both (+)- and (−)-perhexyline inhibited CPT1 activity with similar IC₅₀ values of approximately 50-60 μM. CPT1 activity was measured as described in Kennedy et al. Biochem Pharmacol 52(2): 273-280, 1996.

### Example 6

### Measurement of Perhexyline Concentrations in Plasma, Liver and Heart

Plasma, liver and hear concentrations were measured in Dark Agouti rats treated for 8 weeks with 200 mg/kg/day of (+)-, (−)- or racemic-perhexyline (animals as described in Example 2) using the method of Davies et al., Journal of Chromatography B, 832 (2006) 114-120.

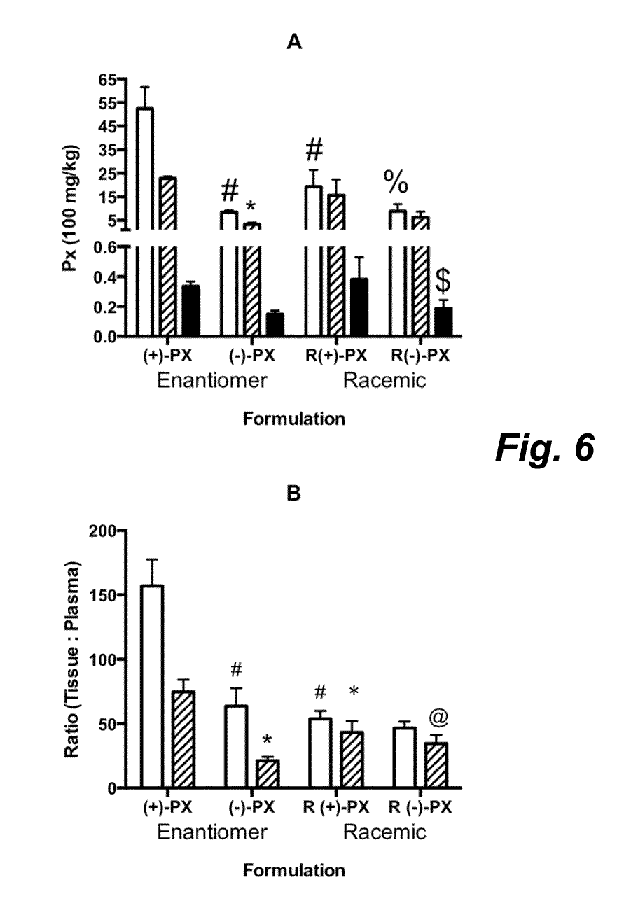

The data are shown in FIGS. 6A and 6B with concentrations adjusted to the equivalent of a 100 mg/kg enantiomeric dose. Panel A shows the dose-adjusted concentrations of (+)- and (−)-perhexyline in liver, heart and plasma following administration of the pure enantiomers and of the racemate, Panel B shows the tissue:plasma concentration ratios for (+)- and (−)-perhexyline in liver and heart, following administration of the pure enantiomers and the racemate.

Measurement of perhexyline concentrations in plasma, liver and heart, shows that whilst tissue distribution of (−)-perhexyline is similar regardless of whether it is administered as pure enantiomer or as part of the racemic formulation, distribution of (+)-perhexyline into liver is significantly increased when administered as the pure enantiomer compared to the racemic formulation, suggesting that for the same myocardial exposure, enantiomerically pure (+)-perhexyline may have a greater potential for hepatotoxicity compared to the racemic formulation.

### Example 7

### Histological Assessment of Myocardial Damage

Despite the studies described above that indicated that (−)-perhexyline produced increased levels of Tropinin T in an isoprenaline rat model, we also investigated the severity and extent of isoprenaline-induced myocardial injury following pre-treatment with racemic perhexyline and each of the enantiomers by histological assessment, as a means to directly assess myocardial damage.

The severity and extent of myocardial injury were assessed by a pathologist who was blinded to the treatment, and classified as: 0—no change; 1—mild (single mild focus of myocyte damage or multiple small foci with mild inflammatory cell infiltrate); 2—moderate (multiple larger foci of myocyte damage with moderate inflammatory cell infiltrate) and 3—severe (multiple larger foci of myocyte damage with severe inflammatory cell infiltrate or broad zone of necrosis with extensive inflammation). Results were analysed by dividing animals into two response groups 0-1: no or mild >1: moderate to severe.

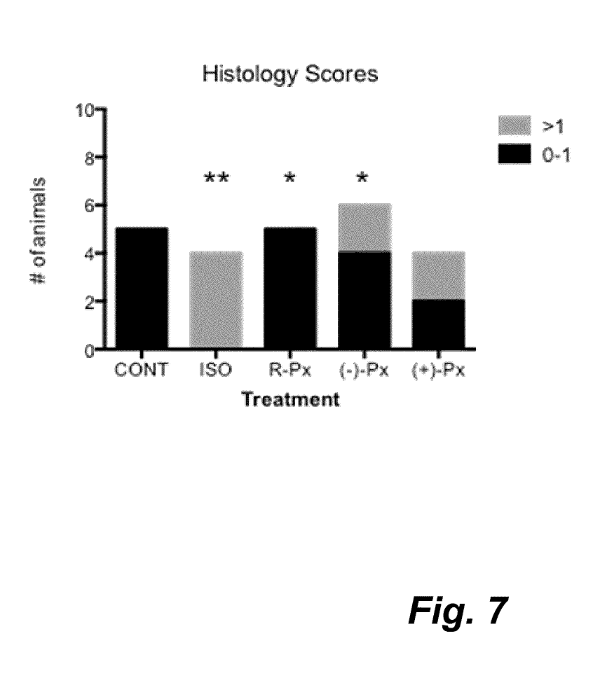

The results are shown in FIG. 7. The results show that isoprenaline treatment caused moderate to severe myocardial inflammation/necrosis as expected. However, contrary to the Tropinin T studies, the damage was also attenuated by (−)-perhexyline, and only partially attenuated by treatment with the (+)-enantiomer.

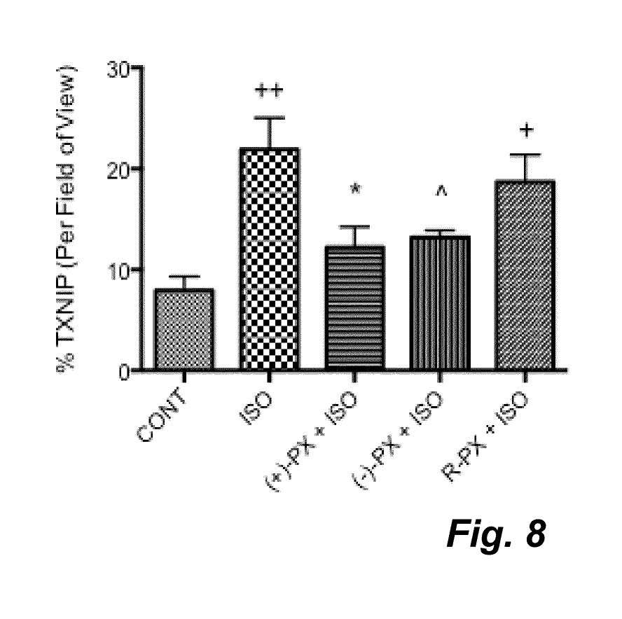

In addition, as shown in FIG. 8, there was a significant increase in myocardial T×NIP staining following isoprenaline injection, which was attenuated by both (+)- and (−)-perhexyline (1-way ANOVA, data shown as mean±sem, assessed by blinded scorer). T×NIP is a regulatory protein which couples substrate utilization and redox state. Increased expression of T×NIP is associated with increased oxidative stress, decreased insulin secretion, decreased glucose uptake and suppression of PPARα (a major nuclear transcription factor regulating lipid metabolism). Thus, both (−)-perhexyline and (+)-perhexyline may also exert cardioprotection via inhibition of T×NIP.

### Example 8

### Activation of Pyruvate Dehydrogenase (PDH)

DA rats were treated for 2 weeks with vehicle or 200 mg/kg/day of racemic, (+)- or (−)-perhexyline maleate, on day 15 myocardial necrosis was induced by i.p. injection of 50 mg/kg isoprenaline (HCl salt), with a second group of controls receiving an i.p. injection of saline (n=5-8). Total and phosphorylated pyruvate dehydrogenase in heart tissue was measured by Western blots.

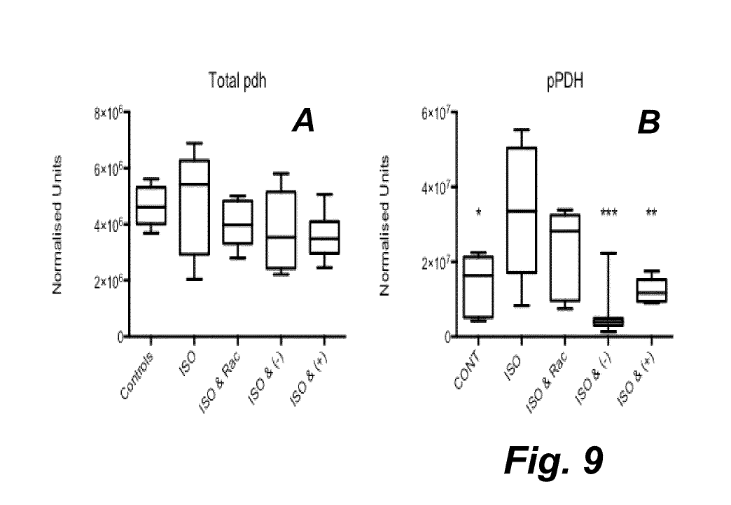

The results are shown in FIG. 9.

(−)-perhexyline did not affect total PDH expression (FIG. 9A), but was an extremely potent inhibitor of PDH deactivaton (measured as phosphorylated PDH) in this model (FIG. 9B). Significantly, it appears to be more potent than (+)-perhexyline, suggesting that the enhancement of carbohydrate utilization by (−)-perhexyline occurs at low concentrations and has a significant component that is independent of CPT1 inhibition. Therefore, the beneficial effects of (−)-perhexyline to enhance myocardial carbohydrate utilization may be dissociated from the potential to cause steatosis and peripheral neuropathy.

### Example 9

### Treatment of Heart Failure, Cardiomyopathy and Ischaemic Heart Disease Using (−)-Perhexyline

Subjects suffering from ischaemic heart disease or heart failure may be identified by known clinical characteristics.

Treatment of human patients with ischaemic heart disease or heart failure may be undertaken by oral administration twice daily of a formulation of 50 mg (−)-perhexyline in tablet form further including lactose, maize starch, sucrose and purified talc or by oral administration once daily of a formulation of 100 mg (−)-perhexyline.

For example, a 50 mg tablet may comprise the following constituents:

(−)-perhexyline maleate: 50 mg as fine white crystalline powder

Lactose

Maize Starch

Sucrose

Talc

Treatment with (−)-perhexyline may be for a defined intervention period (for example 8 weeks) or be maintained indefinitely. Serum and/or plasma (−)-perhexyline levels may be determined at various intervals and any adverse effects monitored. The onset of hepatoxicity, peripheral neuropathy may be monitored. Dose or frequency adjustments can be made based on the serum concentrations, clinical symptoms and any adverse effects.

All methods described herein can be performed in any suitable order unless otherwise indicated herein or otherwise clearly contradicted by context. The use of any and all examples, or exemplary language (e.g., “such as”) provided herein, is intended merely to better illuminate the example embodiments and does not pose a limitation on the scope of the claims appended hereto unless otherwise claimed. No language in the specification should be construed as indicating any non-claimed element as essential.

Although the present disclosure has been described with reference to particular examples, it will be appreciated by those skilled in the art that the disclosure may be embodied in many other forms. Reference to any prior art in this specification is not, and should not be taken as, an acknowledgment or any form of suggestion that this prior art forms part of the common general knowledge in any country. Also, it must be noted that, as used herein, the singular forms “a”, “an” and “the” include plural aspects unless the context already dictates otherwise.

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The subject headings used herein are included only for the ease of reference of the reader and should not be used to limit the subject matter found throughout the disclosure or the claims. The subject headings should not be used in construing the scope of the claims or the claim limitations.

Future patent applications may be filed on the basis of the present application, for example by claiming priority from the present application, by claiming a divisional status and/or by claiming a continuation status. It is to be understood that the following claims are provided by way of example only, and are not intended to limit the scope of what may be claimed in any such future application. Nor should the claims be considered to limit the understanding of (or exclude other understandings of) the present disclosure. Features may be added to or omitted from the example claims at a later date.

What is claimed is:

1. A method for treating or preventing a disease, condition or state in a subject in need thereof, the method comprising administering to the subject an effective amount of the (−)-enantiomer of perhexyline or a pharmaceutically acceptable salt, prodrug or derivative thereof, substantially free of the (+)-enantiomer
wherein the disease, condition or state is associated with a member of the group consisting of: altered tissue energetics, impaired tissue energetics, altered cardiac tissue energetics, impaired cardiac tissue energetics, altered hepatic tissue energetics, impaired hepatic tissue energetics, and diabetes.

2. The method of claim 1, wherein the disease, condition or state is a member selected from the group consisting of: ischaemic heart disease, systolic heart failure, diastolic heart failure, angina, refractory angina, ventricular hypertrophy, congestive cardiomyopathy and hypertrophic cardiomyopathy.

3. The method of claim 1, wherein the disease, condition or state is Type I diabetes or Type II diabetes.

4. The method of claim 1, wherein at least one adverse effect associated with administration of racemic perhexyline is reduced.

5. The method of claim 4, wherein the adverse effect is selected from the group consisting of: hepatotoxicity and neuropathy.

6. The method of claim 1, wherein hepatic glycogen content in the subject is increased without substantially increasing hepatic lipid content.

7. The method of claim 1, wherein hepatic glycogen content in the subject is increased without substantially increasing non-hepatic tissue lipid content.

8. The method of claim 1, wherein at least one therapeutic effect is observed in the subject, the therapeutic effect being selected from a member of the group consisting of: increased glucose utilization, increased myocardial lactate utilization, reduced myocardial lactate accumulation, reduced long chain fatty acid utilization, and increased cardiac efficiency.

9. The method of claim 1, wherein the subject has at least one characteristic selected from the group consisting of: ischaemia, increased tissue NADH/NAD+, reduced tissue pyruvate dehydrogenase activity, increased anaerobic glycolysis, increased fatty acid β-oxidation, reduced phosphocreatine concentration, reduced oxidative phosphorylation, increased insulin resistance, and a reduced ratio of phosphocreatine to ATP.

10. The method of claim 1, wherein the effective amount of (−)-perhexyline administered to the subject produces a plasma concentration in a range selected from a member the group consisting of: 0.05-0.30 mg/L; 0.05-0.60 mg/L; 0.05-0.90 mg/L; 0.05-01.20 mg/L; 0.15-0.30 mg/L; 0.15-0.60 mg/L; 0.15-0.90 mg/L; and 0.15-1.20 mg/L.

11. The method of claim 1, further comprising: determining whether the subject has reduced CYP2D6 activity.

12. The method of claim 1, further comprising: co-administering to the subject at least one member of the group consisting of: an ACE inhibitor, a beta blocker, an aldosterone antagonist, a diuretic, a nitrate, a calcium channel blocker, glucose, insulin, potassium, an insulin sensitiser, and glucagon-like peptide-1.

13. The method of claim 1, wherein the subject is suffering from, or susceptible to, hepatoxicity.

14. The method of claim 1, wherein the subject is suffering from, or susceptible to, peripheral neuropathy.

15. A method for reducing cardiac damage in a subject in need thereof comprising administering to the subject an effective amount of substantially enantiopure (−)-perhexyline or a pharmaceutically acceptable salt, prodrug or derivative thereof.

16. The method of claim 15, wherein the subject is susceptible to, or suffering from, at least one member of the group consisting of: ischaemic heart disease, heart failure, systolic heart failure, diastolic heart failure, angina, refractory angina, ventricular hypertrophy, cardiomyopathy, congestive cardiomyopathy and hypertrophic cardiomyopathy.

17. A pharmaceutical composition comprising substantially enantiopure (−)-perhexyline or a pharmaceutically acceptable salt, prodrug or derivative thereof.

18. The pharmaceutical composition of claim 17, wherein the amount of (−)-perhexyline present is at least 90% of the total perhexyline in the composition.

19. The pharmaceutical composition of claim 17, wherein the composition comprises 25 to 250 mg (−)-perhexyline.

20. The pharmaceutical composition of claim 17, wherein the composition comprises an amount of (−)-perhexyline that when administered to a subject in need thereof once daily produces a plasma concentration in the subject in a range selected from a member of the group consisting of: 0.05-0.30 mg/L; 0.05-0.60 mg/L; 0.05-0.90 mg/L; 0.05-01.20 mg/L; 0.15-0.30 mg/L; 0.15-0.60 mg/L; 0.15-0.90 mg/L; and 0.15-1.20 mg/L.

21. The pharmaceutical composition of claim 17, wherein the composition when administered to a subject in need thereof does not result in substantial hepatoxicity and/or neuropathy.

22. A method for screening for an agent for treating or preventing a disease, condition or state associated with altered tissue energetics, the method comprising:
selecting a modified form of (−)-perhexyline; and
identifying the modified form of (−)-perhexyline as an agent for preventing and/or treating a disease, condition or state associated with altered tissue energetics.

23. A method for screening for a cardiac metabolic agent with reduced hepatotoxicity and/or reduced neuropathy, the method comprising:
selecting a modified form of (−)-perhexyline,
identifying the modified form of (−)-perhexyline as an agent that increases hepatic carbohydrate metabolism without substantially increasing hepatic fatty acid metabolism, and
identifying the modified form of (−)-perhexyline as a cardiac metabolic agent with reduced hepatotoxicity and/or reduced neuropathy.

24. A method for optimizing therapeutic efficacy of (−)-perhexyline, comprising:
administering (−)-perhexyline to a subject in need thereof;
determining a level of the (−)-perhexyline in the subject that is less than a first predetermined level corresponding to a second predetermined amount; and
increasing the amount of (−)-perhexyline subsequently administered to the subject.

25. A method for optimizing therapeutic efficacy of (−)-perhexyline, comprising:
administering (−)-perhexyline to a subject in need thereof;
determining a level of the (−)-perhexyline in the subject that is greater than a first predetermined level corresponding to a second predetermined amount; and
decreasing the amount of (−)-perhexyline subsequently administered to the subject.
